(19) United States
(12) Patent Application Publication
(10) Pub. No.: US 2025/0345468 A1
(43) Pub. Date: Nov. 13, 2025

(54) COMBINATION TREATMENT OF SMALL-CELL LUNG CANCER

(71) Applicant: ITM ISOTOPE TECHNOLOGIES MUNICH SE, Garching/München (DE)

(72) Inventors: Susanne KOSSATZ, München (DE); Wolfgang WEBER, München (DE); Hartmut RAUCH, München (DE); Marian MECKEL, München (DE); Konstantin ZHERNOSEKOV, München (DE)

(21) Appl. No.: 18/860,256

(22) PCT Filed: Apr. 27, 2023
(86) PCT No.: PCT/EP2023/061178

§ 371 Date: Oct. 25, 2024

(30) Foreign Application Priority Data
Apr. 27, 2022 (WO) PCT/EP2022/061254

(51) Int. Cl. A61K 51/08; A61K 31/454; A61K 31/502; A61K 31/5025; A61K 31/55; A61P 35/00; C12Q 1/6886

(52) U.S. Cl. CPC A61K 51/083; A61K 31/454; A61K 31/502; A61K 31/5025; A61K 31/55; A61K 51/088; A61P 35/00; C12Q 1/6886; A61K 2121/00; A61K 2123/00; C12Q 2600/156

(57) ABSTRACT

A combination comprising: (i) a radiopharmaceutical comprising (a) a radionuclide, (b) a chelator, and (c) a Somatostatin receptor binding compound; and (ii) a PARP inhibitor is disclosed. That combination may be used in a method of treating a cancer or an SSTR-positive cancer, such as a neuroendocrine cancer.

### FIELD OF INVENTION

The present invention relates to a combination comprising (i) a radiopharmaceutical comprising a radionuclide, a chelator, a Somatostatin receptor analogue and (ii) a PARP inhibitor suitable for use for the treatment of an SSTR-positive cancer and, more specifically, of a neuroendocrine cancer. The invention further relates to a method of treating an SSTR-positive cancer or a neuroendocrine cancer by a combination comprising (i) a radiopharmaceutical comprising a radionuclide, a chelator, a Somatostatin receptor analogue and (ii) a PARP inhibitor.

### BACKGROUND OF THE INVENTION

The treatment of neuroendocrine tumors is still an area of active research. Neuroendocrine tumors originate from neuroendocrine cells. Neuroendocrine cells are essentially found in all organs of the human body, in particular in the small intestine, the pancreas and the lung bronchioles. They release hormones into the blood in response to a signal from the nervous system. As an example, neuroendocrine tumors of the lung arise from Kulchitzky cells that are normally present in the bronchial mucosa. Accordingly, all neuroendocrine tumors share the common morphologic features of neuroendocrine cells.

Lung cancer is one of the leading causes of cancer-associated mortality worldwide. Lung cancer is a malignant tumor which is characterized by uncontrolled cell growth in the lung tissue. The uncontrolled cell growth allows the cancer to spread beyond the lung tissue, either by direct extension or by entering the lymphatic or hematogenous circulation. This process is referred to as metastasis. Essentially, lung cancers can be classified in two distinct groups, which are subject to different treatment approaches. About 80% of lung cancers are non-small cell lung cancer (NSCLC). NSCLC is further sub-divided into adenocarcinoma, squamous cell carcinoma and large cell carcinoma. Even though these sub-types originate from different lung cell types, they are commonly classified, as their treatment and prognosis are usually similar. About 10% of all lung cancers are referred to as small-cell lung cancer (SCLC). This lung cancer type tends to grow and spread faster than NSCLC. SCLC is strongly associated with exposure to air pollution, smoking and the intake of other airborne noxa.

The majority of SCLC are genetically characterized by bi-allelic inactivation of RB1 (˜90%) and TP53 (˜98%) tumor suppressor genes. The common hypothesis is that inactivation of RB1 in SCLC leads to increase in cellular proliferation due to loss of cell cycle control. Inactivation of TP53 prevents oncogene-induced senescence.

SCLC is typically characterized by neuroendocrine features.

SCLC as an aggressive form of lung cancer is associated with limited therapeutic options. SCLC is usually associated with a high proliferation rate, strong tendency for metastasis and a poor prognosis for the patients affected. Since SCLC tends to grow faster, about two-thirds of the patients are directly diagnosed with extensive-stage SCLC. Despite initial responsiveness to front-line therapy, the overall survival (OS) rate is low—resulting in a high mortality rate. Only app. 6.5% of the affected subjects survive for a period of more than 5-years. An average overall survival period of only 2 to 4 months is reported for patients that are not receiving any active treatment.

Currently, the standard treatment of patients diagnosed with SCLC is a chemotherapeutic approach based on cisplatin and etoposide administration or, less common, based on carboplatin and etoposide.

Another SCLC treatment option is described by EP 3585442 A1. EP 3585442 A1 relates to the treatment of SCLC with therapeutic antibody-drug-conjugates. A drug (SN-38) is attached to an anti-Trop-2 antibody. The administered conjugate can reduce or eliminate metastases and may be effective to treat cancers resistant to standard therapies. That conjugate is considered to be preferably administered in combination with one or more other anti-cancer drugs, such as carboplatin or cisplatin.

WO 2016/207732 A1 relates to methods of treating cancers which over-express somatostatin receptors. Thereby, a combined therapy approach for the treatment of neuroendocrine tumors is realized by a combination of peptide receptor radionuclide therapy (PRRT) and immune-oncologic therapy. The immune-oncologic therapy is based on an inhibitor of the PD-1/PD-L1 pathway.

Despite a larger number of potentially therapeutic approaches, the standard treatment of SCLC is still based on cisplatin or carboplatin. It is widely known that said combination therapy evokes harsh side effects.

A pivotal study reported that enhanced Somatostatin receptor 2 (SSTR2) expression is observed in 50% of advanced SCLC cases. By that study, 46% of the study subjects received radioligand therapy using either ⁹⁰Y-DOTATOC or ¹⁷⁷Lu-DOTATATE. The overall therapeutic efficacy of that radioligand treatment was not convincing. The study authors concluded that lack of promising antitumor effects may be based on a potential radio resistance or the suboptimal tumor-absorbed dose. In summary, it was concluded that despite their potential for precise targeting of SCLC via e.g. SSTR2, the treatment results using radiolabeled SSTR2 agonists, such as ¹⁷⁷Lu-DOTATOC and ¹⁷⁷Lu-DOTATATE as a single agent were not encouraging.

Combination therapies of SSTR targeting and other mechanism were discussed in the literature and are currently under investigation. Pretreatment of SCLC cells with chemotherapeutics, such as gemcitabine, followed by ¹⁷⁷Lu-DOTATATE was reported to be investigated in preclinical phases. In addition, attention is focused on combinations of radioligand therapy and immune-check point inhibitors in an attempt to observe synergistic anti-tumor effects. Recently, a phase I study of ¹⁷⁷Lu-DOTATATE in combination with the anti-PD-1 antibody nivolumab was reported to be well tolerated and to exert antitumor activity in SCLC.

Another class of anti-cancer compounds has been approved for cancer treatment, i.e. PARP inhibitors. The enzyme Poly-(adenosine diphosphate ribose)-Polymerase (PARP) is thus an oncologically attractive target under clinical investigation by administering its specific inhibitors. PARP acts as a responder that detects DNA damage and facilitates the choice of a repair pathway. In particular, PARP is recruited upon DNA single-strand breaks (SSB). In the absence of PARP, DNA replication of SSB compromised DNA leads to DNA double-strand (DSB) breaks, which accumulate due to a destabilized replication fork. As a result, genomic or proteomic deficiencies in the DSB repair pathway of homologous recombination (HR), for example BRCA1/2 mutations, are vulnerable to PARP inhibition. PARP inhibition showed some efficacy in various cancers, such as ovarian, breast, prostate and pancreatic cancer having BRCA1/2 mutations.

CA 2635691 A1 relates to a combination comprising a PARP inhibitor and a cytotoxic agent which may be selected from temozolomide, irinotecan, cisplatin, carboplatin, or topotecan for the treatment of various cancer types.

PARP inhibitor treatment of SCLC, however, was not considered to be effective. Genomic screening of SCLC revealed strong chromosomal rearrangements and a high mutation burden, including inactivation of the tumor suppressor genes TP53 and RB1 and a high level of PARP expression. However, BRCA1/2 mutations were not detected.

Thus, there is still an unmet need for the treatment of SSTR-positive tumors or neuroendocrine tumors and, in particular pulmonary neuroendocrine tumors, such as SCLC as an exceptionally lethal malignancy. Any such treatment should ideally exert strong anti-cancer effects and should invoke less side effects than observed for other therapeutic regimen applied as today's therapy standard.

The present invention is thus directed to the object of treating SSTR-positive cancers or neuroendocrine cancers, in particular pulmonary neuroendocrine cancers.

### SHORT DESCRIPTION OF THE INVENTION

The above-mentioned object is solved by the invention as defined by the claim set. In particular, the problem is solved by a combination comprising as a first component (i) a radiopharmaceutical comprising (a) a radionuclide, (b) a chelator, and (c) a Somatostatin receptor binding compound and as a second component (ii) a PARP inhibitor. The object is also solved by a method for treating an SSTR-positive cancer or a neuroendocrine cancer by the above combination, in particular a pulmonary neuroendocrine cancer. Thus, the combination of the invention comprises two components, (i) the “radiopharmaceutical” and (ii) the “PARP inhibitor”. They are typically provided as two distinct entities, which may separately formulated and separately administered. The radiopharmaceutical is typically composed of a (b) chelator being covalently coupled to the (c) Somatostatin receptor binding compound. The (b) chelator chelates the (a) radionuclide. Optionally, components (i) and (ii) may be further combined with at least one other anti-cancer drug (component (iii)).

The combination according to the present invention may delay tumor growth significantly. The combination according to the invention may also reduce the amount of the radiopharmaceutical, i.e. reduce the radiation, to be administered when combined with a PARP inhibitor for achieving the same level of tumor cell death as observed when administering the radiopharmaceutical alone.

According to the present invention, the radiopharmaceutical of the combination may comprise a radionuclide which is a metal radionuclide. Preferably, it is trivalent metal radionuclide. In one embodiment, it may be selected from the group consisting of ¹⁷⁷Lu, ⁹⁰Y, ⁶⁴Cu, ¹⁶¹Tb, ¹⁸⁸Re, ²²⁵Ac or ²¹³Bi, in particular a 9-particle-emitting radionuclide.

The chelator of the radiopharmaceutical is a macrocyclic chelator, preferably selected from the group consisting of DOTA, HBED-CC, NOTA, NODAGA, DOTAGA, DOTAM, TRAP, NOPO, PCTA and derivatives thereof.

The Somatostatin receptor binding compound of the combination according to the present invention may be a Somatostatin receptor agonist, in particular a peptide or a peptide analogue. The Somatostatin receptor agonist may be selected from the group consisting of TOC, TATE or NOC. DOTA-OC: [DOTA<0>,D-Phe1]octreotide, DOTA-TOC: [DOTA<0>,D-Phe<1>,Tyr1]octreotide (i.e. edotreotide), DOTA-NOC: [DOTA<0>, D-Phe<1>,1-Nal<3>]octreotide, DOTA-TATE: [DOTA<0>,D-Phe′,Tyr<3>]octreotate (i.e. oxodotreotide), DOTA-LAN: [DOTA<0>,D-β-Nal<1>]lanreotide, DOTA-VAP: [DOTA<0>,D-Phe<1>,Tyr<3>]vapreotide, satoreotide trizoxetan, and satoreotide tetraxetan.

A further embodiment according to the present invention may employ a Somatostatin receptor binding compound which is a Somatostatin receptor antagonist. The Somatostatin receptor antagonist may be JR11 or LM3.

The second component (ii) of the combination of the invention is represented by a PARP inhibitor which allows for inhibition of members of the PARP family, e.g. PARP1 and/or PARP2 and/or PARP3. PARP (poly(ADP-ribose) polymerases) are a family of 17 proteins involved in several cellular processes, including stress response, chromatin remodeling, DNA repair and apoptosis. The most well recognized and characterized member of the PARP protein family is PARP1, initially identified for its role in the detection and repair of single-strand DNA breaks (SSBs). More recent evidence suggests that PARP1 may also have a role in alternative DNA repair pathways, including nucleotide excision repair, non-homologous end joining (both classical and alternative), homologous recombination and DNA mismatch repair. DNA damage is rapidly detected through the conserved N-terminal DNA-damage sensing and binding domain of PARP. Subsequently, PARP1 catalyzes the post-translational polymerization of ADP-ribose units (PARs) from NAD⁺ molecules onto target proteins via covalent linkages to acidic residues. PARP2 and PARP3 also have roles in DNA repair processes and share partial redundancy with PARP1 in some of these roles. PARP1, PARP2, and PARP3 share structural similarities and were also shown to be activated in a similar manner through DNA-dependent catalytic activation via local destabilization of the catalytic domain.

For cancer treatment, PARP inhibitors prevent PARP from repairing DNA, e.g. SSBs, in cancer cells and hence support cancer cell death. A PARP inhibitor may be selected from the group consisting of Niraparib, Olaparib, Rucaparib, Talazoparib, Iniparib, Veliparib, Pamiparib, Fluzoparib or Amelparib. More preferably, the PARP inhibitor is selected from the group consisting of Rucaparib, Olaparib, Niraparib and Talazoparib. A PARP inhibitor may not include olaparib. In another embodiment, the group of PARP inhibitors is defined by Rucaparib, Niraparib and Talazoparib. In still another embodiment, the PARP inhibitor is olaparib. One or more PARP inhibitors may be combined as component (ii) of the combination according to the invention. In particular, two PARP inhibitors may be combined as component (ii).

Thereby, the combination according to the present invention may be used for treating a cancer, in particular neuroendocrine cancers, such as pulmonary neuroendocrine cancers, which are classified as small-cell lung cancer (SCLC), carcinoid tumors (typical (TC)/atypical (AC)), and large cell neuroendocrine carcinomas (LCNEC). All of them share common morphological, immunohistochemical and molecular characteristics, which allow them to be commonly classified as neuroendocrine lung tumors.

According to the present invention, the combination may be provided for treating neuroendocrine or pulmonary neuroendocrine cancer patients, such as SCLC cancer patients expressing a Somatostatin receptor, in particular Somatostatin receptor 2, on their cancer cells. Somatostatin receptor expression may be identified by immune histological staining, Somatostatin receptor scintigraphy or positron emission tomography, e.g. as a diagnostic step prior to cancer treatment according to the invention.

Somatostatin Receptor 2 signaling promotes growth and survival in high-grade neuroendocrine lung cancer which supports to target SSTR2 specifically. Studies using somatostatin receptor scintigraphy and positron emission tomography (PET) demonstrated that radiolabeled SSTR2 agonists and antagonists bind precisely to their target. It is known in the art that expression levels in lung cancer derived from neuroendocrine cells are significantly lower than e.g. classical carcinoid tumors originating from the gastrointestinal tract or from the pancreas.

### SHORT DESCRIPTION OF THE FIGURES

The invention is further illustrated by the following figures. However, they are not intended to limit the subject matter of the invention in any way.

FIG. 1 shows a cell viability assay of Rucaparib alone and in combination with ¹⁷⁷Lu-DOTA-TOC. FIG. 1A: H446 and H69 cells treated with increasing concentrations of Rucaparib. FIG. 1B: H446 cells treated with different concentrations of ¹⁷⁷Lu-DOTATOC alone or in combination with Rucaparib (at 5 μM and 20 μM). (IC₅₀ values: ¹⁷⁷Lu-DOTA-TOC: 8.9 kBq, ¹⁷⁷Lu-DOTATOC and 5 μM Rucaparib: 0.9 kBq, ¹⁷⁷LU-DOTATOC and 20 μM Rucaparib; 0.4 kBq)

FIG. 2 shows the quantification of DNA double strand break formation, determined by histone yH2AX foci after single agent treatment (Rucaparib, ¹⁷⁷LU-DOTATOC) and combination treatment of Rucaparib and ¹⁷⁷LU-DOTATOC vs. control (mean with SEM; *p<0.05; Mann-Whitney test, pairwise comparison to all other groups).

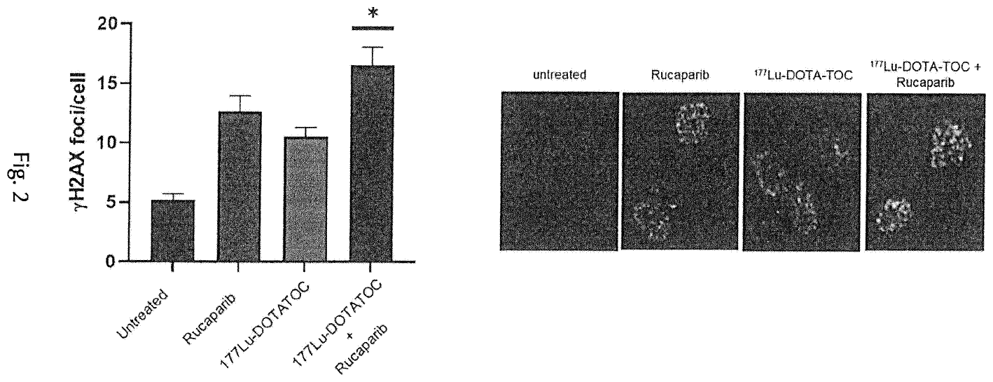

FIG. 3 shows cell viability assays. FIG. 3A: Determination of the IC₅₀ values of Rucaparib in the H446 and H69 cell lines. FIG. 3B: H446, FIG. 3C: H69 cell lines. Cell viability was studied upon combined cell treatment with Olaparib and ¹⁷⁷Lu-DOTA-TOC vs. ¹⁷⁷Lu-DOTA-TOC alone. In both cell lines a lower amount of ¹⁷⁷Lu-DOTATOC was determined when applying combined treatment than for ¹⁷⁷Lu-DOTATOC alone to have the same cell killing effect.

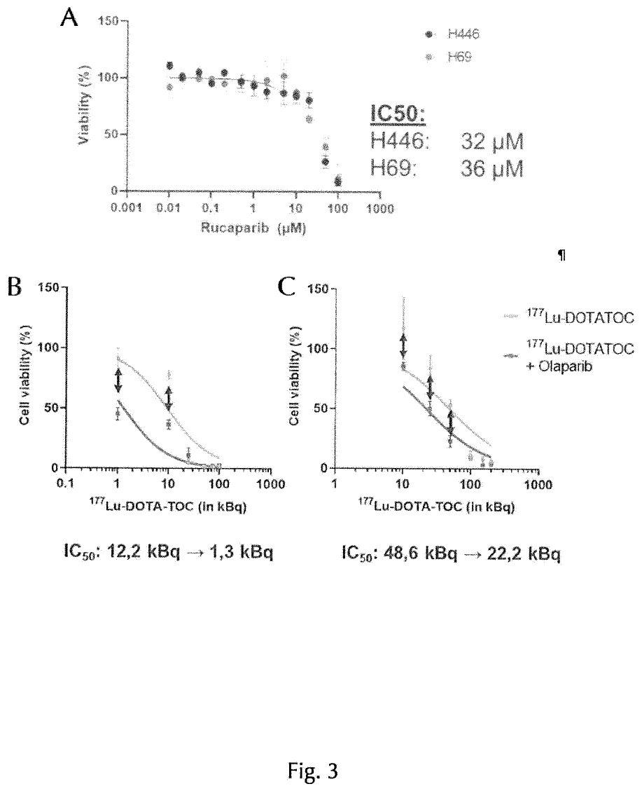

FIG. 4 shows cell viability assays in H446 (FIG. 4A) and H69 (FIG. 4B) cell lines. Cell viability was studied upon combined cell treatment with Talazoparib and ¹⁷⁷Lu-DOTA-TOC vs. ¹⁷⁷Lu-DOTA-TOC alone. In both cell lines a lower amount of ¹⁷⁷Lu-DOTATOC was determined when applying combined treatment than for ¹⁷⁷Lu-DOTATOC alone to have the same cell killing effect.

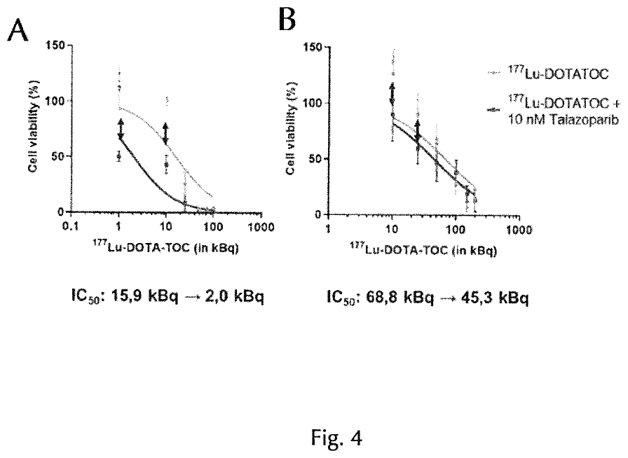

FIG. 5 shows a tumor growth delay curve (FIG. 5A; mean tumor volume in mm³) and Kaplan-Meier survival plot (FIG. 5B) of H446 tumor cell bearing mice treated with ¹⁷⁷Lu-DOTATOC, Rucaparib and the combination thereof (and the negative control: untreated mice). The underlying protocol is depicted by FIG. 10. Arrow depicts the application of radiopharmaceutical treatment. White boxes depict the PARP inhibitor treatment duration. Tumor growth is significantly delayed by the combined treatment (p>0.01).

FIG. 6 shows the tumor growth delay curve (FIG. 6A; tumor volume in mm³) and Kaplan-Meier survival plot (FIG. 6B) of H69 tumor bearing mice treated with ¹⁷⁷Lu-DOTATOC, Rucaparib, or Olaparib, respectively alone and combinations of ¹⁷⁷Lu-DOTATOC with Rucaparib or Olaparib (and the negative control: untreated mice). The underlying protocol is depicted by FIG. 11. Arrow depicts the application of radiopharmaceutical treatment. White boxes depict the PARP inhibitor treatment duration. Tumor growth is significantly delayed by the combined treatment approaches. At the end of the experiment 4/6 animals of the ¹⁷⁷Lu-DOTATOC/Rucaparib treated group and all 6 animals of the ¹⁷⁷Lu-DOTATOC/Olaparib treated group were still alive.

FIG. 7 depicts the results of the body weight monitoring of tumor bearing mice undergoing PARP inhibitor treatment (Rucaparib, or Olaparib), ¹⁷⁷Lu-DOTATOC, and combined radiopharmaceutical/PARP inhibitor treatment.

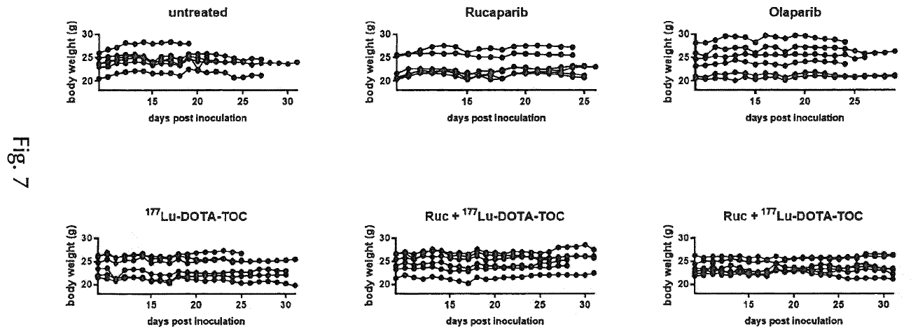

FIG. 8 shows organ distribution of 10 MBq ⁶⁸Ga-DOTATOC (% ID/g: percentage of injected dose per gram of tissue) 4 hours p.i. H446 and H69 as SSTR2 expressing SCLC cell lines were subcutaneously inoculated into mice to establish a mouse xenograft model. The results were obtained from PET scans. The measured signal is observed as an uptake in tumor tissue and in the kidneys, with minor signals in the gastrointestinal tract and the pancreas. By control experiments, the tumors were blocked with non-radioactive (non-labelled) DOTATOC (octreotide) for comparative reasons (“w/Block”).

FIG. 9 depicts PET images (maximum intensity projection of ⁶⁸Ga-DOTATOC)) of the untreated control group, the group treated with Rucaparib alone, the group treated with ¹⁷⁷Lu-DOTATOC alone, combination treatment (at day 8) and combination treatment at day 15. The mice used for that experiment are H446 cell tumor bearing mice. The images show the presence of the SSTR2-positive cells with no significant change of their expression levels. Tumor tissue is indicated as “T”, the kidney as “Ki” and the bladder as “BI”.

FIG. 10 depicts the mouse trial protocol as employed for the experimental set-up underlying rucaparib administration (monotherapy), ¹⁷⁷Lu-DOTATOC (monotherapy), untreated control group and the combinatorial approach according to the invention based on rucaparib in combination with ¹⁷⁷Lu-DOTATOC (with the results of that experiment shown in FIG. 5).

FIG. 11 depicts the mouse trial protocol as employed for the experimental set-up underlying rucaparib and olaparib administration (each as monotherapy), ¹⁷⁷Lu-DOTATOC (monotherapy), untreated control group and the combinatorial approach according to the invention based on either rucaparib or olaparib in combination with ¹⁷⁷Lu-DOTATOC (with the results of that experiment shown in FIG. 6).

### DETAILED DESCRIPTION OF THE INVENTION

The invention relates to a two-component combination of (i) a radiopharmaceutical comprising a (a) radionuclide, (b) a chelator and (c) a somatostatin receptor binding compound and (ii) a PARP inhibitor, in particular for use in a method of treating a cancer or an SSTR-positive cancer or of a neuroendocrine cancer. It relates also to a method of treating a cancer or an SSTR-positive cancer, in particular of a neuroendocrine cancer.

As used herein, the term “combination” refers to any kind of combination of its components, in particular, to any kind of combination of (i) the radiopharmaceutical and (ii) the PARP inhibitor and, optionally, any further components. In particular, the components of a combination are provided and/or administered in a combined mode according to a treatment protocol such that they may display their advantageous therapeutic profile resulting from their combined action on the tumor. In some embodiments, the combination may be a kit (e.g., comprising the components in an (at least partially) separated manner). In other embodiments, the combination may be a composition (e.g., the components may be comprised in one single composition).

The two-component combination of the present invention is characterized by an improved anti-tumor effect resulting from radiation exposure by the radiopharmaceutical component and from enhanced by DNA damage repair (DDR) inhibition by the PARP inhibitor compound, acting as a radiosensitizer. When using PARP inhibitors or e.g. ¹⁷⁷Lu-DOTATOC alone, the resulting anti-tumor effect is significantly less pronounced than by the combination of said components, which act commonly based on a synergistic mode of action.

The treatment of neuroendocrine tumors is typically not straight-forward. In particular, the treatment of SCLC as of today is essentially palliative due to its late stage profile when diagnosed. The present invention, however, may allow to treat even late stage neuroendocrine cancer patients in a curative manner, e.g. late stage cancer patients suffering from SCLC. “Late stage neuroendocrine cancer patients” may be characterized by tumor cells spread to the lymph nodes and, potentially, other organs. Targeted radioligand therapy according to the present invention implies the delivery of relatively high radiation doses to even small lesions and distant metastases. Moreover, healthy tissue is not damaged by the specificity of the radiopharmaceutical for tumor target cells expressing a Somatostatin receptor on their cell surface, in contrast to e.g. beam radiation therapy. The combination according to the invention was found to exhibit a higher level of therapeutic efficacy and to have less or at least acceptable side effects when treating neuroendocrine tumors, in particular when treating SCLC.

Thus, the present inventors identified radioligand therapy in combination with PARP inhibitor administration as an effective and well tolerated treatment of an SSTR-positive cancer or of a neuroendocrine cancer, in particular for patients suffering from SCLC.

### General Comments

Although the present invention is described in detail below, it is to be understood that this invention is not limited to particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by the skilled person in the art.

In the following, the elements of the present invention will be described. These elements are listed with specific embodiments, however, it should be understood, that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The term “about” in relation to a numerical value x means x±10%.

The term “subject” as used herein generally includes humans and non-human animals and preferably mammals (e.g., non-human primates, including marmosets, tamarins, spider monkeys, owl monkeys, vervet monkeys, squirrel monkeys, and baboons, macaques, chimpanzees, orangutans, gorillas; cows; horses; sheep; pigs; chicken; cats; dogs; mice; rat; rabbits; guinea pigs etc.), including chimeric and transgenic animals and disease models, in particular humans.

As used herein, “safe” and “effective” amounts mean an amount of agents that is sufficient to allow for diagnosis and/or significantly induce a positive modification of the disease to be treated. At the same time, however, a “safe” and “effective” amount is small enough to avoid serious side-effects, that is to say permitting a sensible relationship between advantage and risk. A “safe” and “effective” amount will furthermore vary in connection with the particular condition to be diagnosed or treated and also with the age and physical condition of the patient to be treated, the severity of the condition, the duration of the treatment, the nature of the accompanying therapy, of the particular pharmaceutically acceptable excipient or carrier used, and similar factors.

### (A) Radiopharmaceutical as Component (i) of the Combination

The radiopharmaceutical as a component of the combination of the invention comprises (a) a radionuclide, (b) a chelator, and (c) a somatostatin receptor binding compound. (a), (b) and (c) typically form a (complexed) conjugate molecule. Their salts, solvates or tautomers are included as well.

### (a) Radionuclide of the Radiopharmaceutical

The term “radionuclide” (or “radioisotope”) refers to isotopes of natural or artificial origin with an unstable neutron to proton ratio that disintegrates with the emission of corpuscular (i.e. proton (alpha-radiation) or electron (beta-radiation)) or electromagnetic radiation (gamma-radiation)). In other words, radionuclides undergo radioactive decay. In the radiolabeled complex of the radiopharmaceutical as one component of the two-component combination of the invention, any known radionuclide suitable for therapy may be complexed by the chelating agent. Such radionuclides may include, without limitation, ¹³¹I, ⁹⁴Tc, ^{99m}Tc, ⁹⁰In, ¹¹¹In, ⁶⁷Ga, ⁶⁸Ga, ⁸⁶Y, ⁹⁰Y, ¹⁷⁷Lu, ¹⁶¹Tb, ¹⁸⁶Re, ¹⁸⁸Re, ⁶⁴Cu, ⁶⁷Cu, ⁵⁵Co, ⁵⁷Co, ⁴³Sc, ⁴⁴Sc, ⁴⁷Sc, ²²⁵Ac, ²¹³Bi, ²¹²Bi, ²¹²Pb, ²²⁷Th, ¹³³Sm, ¹⁶⁶Ho, ¹⁵²Gd, ¹⁵³Gd, ¹⁵⁷Gd, or ¹⁶⁶Dy, in particular selected from the group consisting of ⁶⁸Ga, ¹⁷⁷Lu, ²²⁵Ac, ¹⁶¹Tb, ²¹³Bi, ¹⁸⁸Re, ⁶⁴Cu and ⁹⁰Y or the group consisting of ⁶⁸Ga, ¹⁷⁷Lu and ⁹⁰Y or the group consisting of ^{99m}Tc, ¹¹¹In, ⁹⁰Y, and ¹⁷⁷Lu. In one embodiment, the radionuclide is ¹⁷⁷Lu. Typically, the radionuclide is a β-particle emitting radionuclide converting a neutron to a proton by electron emission. It is typically a metal radionuclide, preferably a trivalent metal radionuclide.

The choice of suitable radionuclides for the provision of a radiopharmaceutical of the inventive combination depends inter alia on the chemical structure and chelating capability of the chelator, and, most prominently, on the intended application of the resulting (complexed) conjugate molecule. For instance, the beta-emitters such as ⁹⁰Y, ¹³¹I, ¹⁶¹Tb and ¹⁷⁷Lu may be used for concurrent systemic radionuclide therapy according to the present invention. Providing DOTA, DOTAGA or DOTAM as a chelator may advantageously enable the use of either ⁶⁸Ga, ^{43, 44, 47}Sc, ¹⁷⁷Lu, ¹⁶¹Tb, ²²³Ac, ²⁷³Bi, or ²¹²Pb as radionuclides. In some preferred embodiments, the radionuclide may be ¹⁷⁷Lu. In other preferred embodiments, the radionuclide may be ⁹⁰Y. In another preferred embodiments, the radionuclide may be ⁶⁴Cu. In some preferred embodiments, the radionuclide may be ¹⁶¹Tb.

### (b) Chelator of the Radiopharmaceutical

The chelating agent or a chelator of the radiopharmaceutical allows for coordination of the radionuclide. Moreover, the chelator or chelating agent is advantageously covalently linked to the (c) Somatostatin receptor binding compound. The chelator group, for example the DOTA group, chelates a central (metal) radioisotope, in particular a radionuclide as specifically defined herein for forming the radiopharmaceutical of the combination of the invention.

The terms “chelator” or “chelating agent” are used interchangeably herein. They refer to polydentate (multiple bonded) ligands capable of forming two or more separate coordinate bonds with (“coordinating”) a central (metal) ion. Specifically, such molecules or molecules sharing one electron pair may also be referred to as “Lewis bases”. The central (metal) ion is usually coordinated by two or more electron pairs to the chelating agent. The terms, “bidentate chelating agent”, “tridentate chelating agent”, and “tetradentate chelating agent” are known to the skilled person and refer to chelating agents having, respectively, two, three, and four electron pairs readily available for simultaneous donation to a metal ion coordinated by the chelating agent. Usually, the electron pairs of a chelating agent forms coordinate bonds with a single central (metal) ion; however, in certain examples, a chelating agent may form coordinate bonds with more than one metal ion, with a variety of binding modes being possible. The terms “coordinating” and “coordination” refer to an interaction in which one multi electron pair donor coordinatively bonds (is “coordinated”) to, i.e. shares two or more unshared pairs of electrons with, preferably one central (metal) ion. The chelator or chelating agent is preferably a macrocycle. More preferably, the chelator is a macrocyclic bifunctional chelator having a metal chelating group at one end and a reactive functional group at the other end, which is capable to be linked to other moieties, e.g. peptides, such as Somatostatin receptor binding compounds. Preferably, the chelator may be selected such that the chelator forms a square bi-pyramidal complex for complexing the radionuclide. In another embodiment, the chelator does not form a planar or a square planar complex. The chelating agent is preferably chosen based on its ability to coordinate the desired central (metal) ion, which is a radionuclide as specified herein.

Preferably, the chelator may be DOTA (1,4,7,10-tetraazacyclododecane-1,4,7,10-tetraacetic acid), HBED-CC (N,N″-bis[2-hydroxy-5-(carboxyethyl)benzyl]ethylenediamine-N,N″-diacetic acid), DOTAGA (2-[1,4,7,10-Tetraazacyclododecane-1,4,7,10-tris(acetate)]-pentanedioic acid), DOTAM (1,4,7,10-Tetrakis(carbamoylmethyl)-1,4,7,10-tetraazacyclododecane) or derivatives thereof. Advantageously, DOTA effectively forms complexes with diagnostic and therapeutic (e.g. ⁹⁰Y or ¹⁷⁷Lu) radionuclides and thus enables the use of the same conjugate radiopharmaceutical for both imaging (diagnostic) and therapeutic purposes, i.e. as a theragnostic agent. DOTA derivatives capable of complexing Scandium radionuclides (^{43, 44, 47}Sc), including DO3AP, DO3AP^{PrA}, or DO3^{ABn} may also be preferred and are described in Kerdjoudj et al. (Dalton Trans., 201 6, 45, 1398-1409).

Other preferred chelators in the context of the present invention include, (2-(4,7-bis(carboxymethyl)-1,4,7-triazonan-1-yl)-pentanedioic acid (NODAGA), 1,4,7-triazacyclo-nonane-1,4,7-triacetic acid (NOTA), 2-(4,7,10-tris(carboxymethyl)-1,4,7,10-tetra-azacyclododecan-1-yl)-pentanedioic acid (DOTAGA), 1,4,7-triazacyclononane phosphinic acid (TRAP), 1,4,7-triazacydo-nonane-1-[methyl(2-carboxyethyl)-phosphinic acid]-4,7-bis-[methyl-(2-hydroxymethyl)-phosphinic acid](NOPO), 3,6,9,15-tetra-azabicyclo[9.3.1]-pentadeca-1(15),11,13-triene-3,6,9-triacetic acid (PCTA), N′-{5-[acetyl(hydroxy)amino]-pentyl}-N-[5-({4-[(5-aminopentyl)(hydroxy)amino]-4-oxobutanoyl}-amino)pentyl]-N-hydroxy-succinamide (DFO), diethylene-triaminepentaacetic acid (DTPA), and hydrazinonicotinamide (HYNIC).

For instance, in some preferred embodiments, the chelator may be DOTA and the radionuclide may be ¹⁷⁷Lu. In other preferred embodiments, the chelator may be DOTA and the radionuclide may be ⁶⁸Ga. In other preferred embodiments, the chelator may be HYNIC and the radionuclide may be ^{99m}Tc.

### (c) Somatostatin Receptor Targeting Compound of the Radiopharmaceutical

The radiopharmaceutical component (i) of the combination according to the invention comprises a compound targeting the somatostatin receptor on cancer target cells. Such a targeting compound of the radiopharmaceutical may be preferably a peptide or a peptide analog. It is preferably covalently linked to (b) the chelator. The Somatostatin receptor binding compounds may be structurally diverse, but are functionally typically somatostatin analogs. Typically, the targeting Somatostatin receptor binding peptide or peptide analog has a cyclic basic structure by forming an intramolecular disulfide bridge established by the side chains of two cystein residues. Their salts, solvates or tautomers are included by the present invention as well.

Peptides targeting the somatostatin receptor may be selected from the group consisting of somatostatin analogues tyr3-octreotide (D-Phe-c(Cys-Tyr-D-Trp-Lys-Thr-Cys)-Thr(ol)), tyr3-octeotrate (D-Phe-c(Cys-Tyr-D-Trp-Lys-Thr-Cys)-Thr) (Capello A et al.: Tyr3-octreotide and Tyr3-octreotate radiolabeled with ¹⁷⁷Lu or ⁹⁰Y: peptide receptor radionuclide therapy results in vitro, Cancer Biother Radiopharm, 2003 Oct.; 1 8(5): 761-8), octreotide (D-Phe-cyclo(Cys-Phe-D-Trp-Lys-Thr-Cys)Thr(ol)), and NOC (D-Phe-cyclo(Cys-1-Nal-D-Trp-Lys-Thr-Cys)Thr(ol)).

Said somatostatin receptor binding compound may be preferably selected from a peptide or a peptide analog of the group consisting of octreotide, octreotate, lanreotide, vapreotide, pasireotide, ilatreotide, pentetreotide, depreotide, satoreotide, veldoreotide. Even more preferably, the targeting molecule is a somatostatin receptor binding compound selected from octreotide and octreotate.

Others examples of compounds targeting the somatostatin-receptor are somatostatin antagonistic peptides such as JR10 (p-NO₂-Phe-c(D-Cys-Tyr-D-Aph(Cbm)-Lys-Thr-Cys)D-Tyr-NH₂); JR11 (Cpa-c(D-Cys-Aph(Hor)-d-Aph(Cbm)-Lys-Thr-Cys)D-Tyr-NH₂); BASS (p-NO₂-Phe-cyclo(D-Cys-Tyr-D-Trp-Lys-Thr-Cys)D-Tyr-NH₂; and LM3 (p-Cl-Phe-cyclo(D-Cys-Tyr-D-Aph(Cbm)-Lys-Thr-Cys)D-Tyr-NH₂.

Somatostatin-receptor binding peptides as disclosed herein may be combined with a chelator as disclosed herein. Hereby, DOTATE (DOTA-(Tyr3)-octreotate), DOTATOC (DOTA-[Tyr3]-octreotide), DOTANOC (DOTA-D-Phe-cyclo(Cys-1-Nal-D-Trp-Lys-Thr-Cys)Thr(ol)), DOTA-JR11 or DOTA-LM3 may be preferably employed.

According to another embodiment, an antagonist as a receptor binding compound of (i) the radiopharmaceutical may be selected from the group consisting of:
(i) pNO₂-Phe-cyclo[D-Cys-Tyr-D-Trp-Lys-Thr-Cys]-D-Tyr-NH₂;
(ii) pNO₂-Phe-cyclo[D-Cys-Tyr-D-Aph(Cbm)-Lys-Thr-Cys]-D-Tyr-NH₂;
(iii) H₂N-pNO₂-Phe-cyclo[D-Cys-Tyr-D-Aph(Cbm)-Lys-Thr-Cys]-2Nal-NH₂;
(iv) pNO₂-Phe-cyclo[D-Cys-Tyr-D-Aph(Cbm)-Lys-Thr-Cys]-2Nal-NH₂;
(v) pNO₂-Phe-cyclo[D-Cys-Aph(Hor)-D-Aph(Cbm)-Lys-Thr-Cys]-2Nal-NH₂;
(vi) Cpa-cyclo[D-Cys-L-Agl(NMe.benzoyl)-D-Trp-Lys-Thr-Cys]-2Nal-NH₂;
(vii) Cpa-cyclo[D-Cys-D-Agl(NMe.benzoyl)-D-Trp-Lys-Thr-Cys]-2Nal-NH₂;
(viii) Cpa-cyclo[D-Cys-Leu-D-Trp-Lys-Thr-Cys]-2Na-NH₂;
(ix) Cpa-cyclo[D-Cys-Aph(Cbm)-D-Trp-Lys-Thr-Cys]-2Nal-NH₂;
(x) Cbm-Cpa-cyclo[D-Cys-Aph(Cbm)-D-Trp-Lys-Thr-Cys]-2Nal-NH₂;
(xi) [beta]Ala-Cpa-cyclo[D-Cys-Aph(Cbm)-D-Trp-Lys-Thr-Cys]-2Nal-NH₂;
(xii) Cpa-cyclo[D-Cys-Aph(Cbm)-D-Trp-Lys-Thr-Cys]-2Nal-NH₂;
(xiii) Cpa-cyclo[D-Cys-Aph(Cbm)-D-Trp-Lys-Thr-Cys]-NH₂;
(xiv) Cpa-cyclo[D-Cys-Aph(Cbm)-D-Trp-Lys-Thr-Cys]-Cha-NH₂;
(xv) Cpa-cyclo[D-Cys-Aph(Cbm)-D-Trp-Lys-Thr-Cys]-Aph(Hor)-NH₂;
(xvi) Cpa-cyclo[D-Cys-Aph(Cbm)-D-Trp-Lys-Thr-Cys]-DAph(Cbm)-NH₂;
(xvii) Cpa-cyclo[D-Cys-Aph(Cbm)-D-Trp-Lys-Thr-Cys]-Aph(Cbm)-NH₂;
(xviii) Cpa-cyclo[D-Cys-Aph(Cbm)-D-Trp-Lys-Thr-Cys]-D-Aph(Cbm)-GlyOH;
(xix) Cpa-cyclo[D-Cys-Aph(CONH—OCH3)-D-Trp-Lys-Thr-Cys]-2Nal-NH₂;
(xx) Cpa-cyclo[D-Cys-Aph(CONH—OH)-D-Trp-Lys-Thr-Cys]-2Nal-NH₂;
(xxi) Cpa-cyclo[D-Cys-Aph(Cbm)-5F-D-Trp-Lys-Thr-Cys]-2Nal-NH₂;
(xxii) Cpa-cyclo[D-Cys-Aph(Cbm)-5F-Trp-Lys-Thr-Cys]-2Nal-NH₂;
(xxiii) Cpa-cyclo[D-Cys-Tyr-D-Aph(Cbm)-Lys-Thr-Cys]-2Nal-NH₂;
(xxiv) Cpa-cyclo[D-Cys-Aph(Hor)-D-Aph(Cbm)-Lys-Thr-Cys]-2Nal-NH₂;
   and
(xxv) Cpa-cyclo[D-Cys-Aph(Hor)-D-Aph(Cbm)-Lys-Thr-Cys]-D-Tyr-NH₂.
   or salts, solvates or tautomers thereof.

### (d) Radiopharmaceutical and Radiopharmaceutical Composition

The radiopharmaceutical is composed of (a) the radionuclide, (b) the chelator and (c) the Somatostatin receptor binding compound. Typically, the radiopharmaceutical represents one single entity.

Examples for the radiopharmaceutical of the combination according to the invention may include or may be selected from the group consisting of: ⁷⁷Lu-DOTATOC (¹⁷⁷Lu-DOTA-[Tyr3]-octreotide), ¹⁷⁷Lu-DOTA-D-Phe-cyclo(Cys-Tyr-D-Trp-Lys-Thr-Cys]-Thr(ol), ¹⁷⁷Lu-DOTANOC (¹⁷⁷Lu-DOTA-D-Phe-cyclo(Cys-1-Nal-D-Trp-Lys-Thr-Cys)Thr(ol)), ¹⁷⁷Lu-DOTATATE (¹⁷⁷Lu-DOTA-D-Phe-cyclo(Cys-Tyr-D-Trp-Lys-Thr-Cys)Thr), ⁶⁸Ga-DOTATOC (⁶⁸Ga-DOTA-D-Phe-cyclo(Cys-Tyr-D-Trp-Lys-Thr-Cys)Thr(ol)), ⁶⁸Ga-DOTANOC (⁶⁸Ga-DOTA-D-Phe-cyclo(Cys-1-Nal-D-Trp-Lys-Thr-Cys)Thr(ol)), ⁹⁰Y-DOTATOC (⁹⁰Y-DOTA-D-Phe-cyclo(Cys-Tyr-D-Trp-Lys-Thr-Cys)Thr(ol)), ⁹⁰Y-DOTATATE (⁹⁰Y-DOTA-D-Phe-cycloiCys-Tyr-D-Trp-Lys-Thr-Cys)Thr), and ¹¹¹In-DTPA-octreotide (¹¹¹In-DTPA-D-Phe-cyclo(Cys-Phe-D-Trp-Lys-Thr-Cys)Thr(ol)). ¹⁷⁷Lu-DOTATOC (¹⁷⁷Lu-DOTA-[Tyr3]-octreotide) may be preferred. Other examples of the radiopharmaceutical of the invention may include or may be selected from the group consisting of: ¹¹¹In-DOTA-BASS (¹¹¹In-DOTA-p-NO₂-Phe-cyclo-(D-Cys-Tyr-D-Trp-Lys-Thr-Cys)D-Tyr-NH₂, ¹¹¹In-DOTA-JR11 (¹¹¹In-DOTA-Cpa-cyclo[D-Cys-Aph(Hor)-D-Aph(Cbm)-Lys-Thr-Cys]DD-Tyr-NH₂), ⁶⁸Ga-DOTA-JR11 (Ga-OpS201) (⁶⁸Ga-DOTA-Cpa-cyclo[D-Cys-Aph(Hor)-D-Aph(Cbm)-Lys-Thr-Cys]D-Tyr-NH₂), ⁶⁸Ga-DODAGA-JR11 (Ga-OPS202) (⁶⁸Ga-NODAGA-Cpa-cyclo[D-Cys-Aph(Hor)-D-Aph(Cbm)-Lys-Thr-Cys]D-Tyr-NH₂), and ¹⁷⁷Lu-DOTA-JR11 (Lu-OPS201) (¹⁷⁷Lu-DOTA-Cpa-cyclo[D-Cys-Aph(Hor)-D-Aph(Cbm)-Lys-Thr-Cys]D-Tyr-NH₂). The above radiopharmaceuticals may be modified by replacing the radionuclide defined above by another radionuclide, e.g. by replacing ¹⁷⁷Lu, ¹¹¹In or ⁶⁸Ga by another radionuclide, in particular a radionuclide as disclosed herein, e.g. by ⁹⁰Y or ¹⁶¹Tb.

In another embodiment, two distinct, e.g. two of the above listed radiopharmaceuticals may be combined as component (i) of the combination of the invention, e.g. ¹⁷⁷Lu-DOTATOC and ⁹⁰Y-DOTATATE, either by one single radiopharmaceutical composition or, preferably, by two distinct radiopharmaceutical compositions.

More generally, the Somatostatin receptor binding peptide antagonists or peptide antagonist, chelator conjugates or radiopharmaceuticals as described herein may form solvates with water (such as hydrates or hemihydrates) or common organic solvents. The term “tautomer” as used herein is used in its broadest sense and includes peptides or peptide/chelator conjugates or radiopharmaceuticals of the present invention which are capable of existing in a state of equilibrium between two isomeric forms. Such compounds may differ in the bond connecting two atoms or groups and the position of these atoms or groups in the compound.

Radiopharmaceuticals may be administered in the form of pharmaceutically or veterinarily acceptable non-toxic salts, such as acid addition salts or metal complexes, e.g., with zinc, iron, calcium, barium, magnesium, aluminum or the like. Such non-toxic salts may be hydrochloride, para-toluenesulfonate, hydrobromide, sulphate, phosphate, tannate, oxalate, fumarate, gluconate, alginate, maleate, acetate, citrate, benzoate, succinate, malate, ascorbate, tartrate and the like.

The radiopharmaceutical is typically provided and administered as a radiopharmaceutical composition, comprising the radiopharmaceutical as described herein and at least one pharmaceutically acceptable excipient. More specifically, the pharmaceutical composition may be a liquid formulation, e.g. for systemic administration, e.g. intravasal or intravenous administration by injection or infusion. It may be an aqueous solution, optionally containing a buffer system. The aqueous pharmaceutical composition of the invention may comprise another water-miscible (organic) solvent, e.g. ethanol. Typically, the aqueous solution may not contain more than 10% of another solvent, e.g. ethanol, by volume. The pharmaceutical composition may have a pH in the range of pH 3 to pH 7, more specifically in the range of pH 3.5 to pH 6 or pH 4 to pH 6.

The radiopharmaceutical composition comprises 0.001 to 1 mg/ml radiopharmaceutical as defined herein, depending on the subject to be treated or the disease to be treated. Specifically, the concentration of the radiopharmaceutical may be in the range of 0.01 to 1 or 0.5 mg/ml or 0.05 to 0.5 mg/ml.

The radiopharmaceutical composition may contain at least one, e.g. 1, 2 or 3 of the additives of the group consisting of gentisic acid, ethanol, acetate, NaCl and ascorbic acid/ascorbate.

In one embodiment, the radiopharmaceutical composition comprises the antioxidant ascorbic acid/ascorbate as a stabilizer. The presence of ascorbic acid/ascorbate, which typically acts as scavengers, may stabilize the radiopharmaceutical composition, thereby enhancing the shelf life of the radiopharmaceutical composition, while maintaining the radiopharmaceutical composition as being suitable for administration to a human, and other mammalian subjects. The radiopharmaceutical composition may thus comprise greater than about 5 mg of ascorbic acid per milliliter or greater than about 10 mg of ascorbic acid per milliliter or greater than about 20 mg of ascorbic acid per milliliter or greater than about 30 mg of ascorbic acid per milliliter or greater than about 40 mg of ascorbic acid per milliliter or greater than about 50 mg of ascorbic acid per milliliter or greater than about 100 mg of ascorbic acid per milliliter or greater than about 200 mg of ascorbic acid per milliliter. The radiopharmaceutical composition may thus contain 5 to 100 mg/ml of ascorbic acid/ascorbic acid or 25 to 500 mg/ml or 50 to 200 mg/ml. To put it differently, the radiopharmaceutical composition may comprise ascorbic acid/ascorbate in the range of 0.5 mM to 0.5 M, in particular 1 mM to 100 mM or 10 mM to 100 mM.

The radiopharmaceutical composition may exhibit a radioactivity in the range of 50 to 800 MBq/ml or 100 to 600 MBq/ml or 200 to 400 MBq/ml.

The radiopharmaceutical may be selectively accumulated in the tumor tissue of a tumor patient in vivo. Thereby, the radiopharmaceutical accumulates in a much less pronounced manner in tissue other than tumor tissue. Preferential accumulation within the tumor tissue may be expressed by the ratio of radiopharmaceutical uptake in tumor cells to radiopharmaceutical uptake in other tissues, e.g. kidney, liver, or blood. The tumor/blood uptake ratio characterizing an inventive radiopharmaceutical may be at least 50.0 or at least 75 or at least 100. The tumor/liver uptake ratio of radiopharmaceutical may be at least 10.0 or at least 20. The ratio may be determined as shown in the biodistribution in vivo studies (section III.) below. The tumor/kidney uptake ratio may be at least 2.0. That measurement may be carried at least 2 hours after administration of the radiopharmaceutical to a subject, typically 2 or 4 hours after administration of the radiopharmaceutical to the subject.

### (B) PARP Inhibitor as Component (ii) of the Combination

The combination according to the present invention comprises a PARP inhibitor. PARP inhibitors are small organic compounds that are able to inhibit the enzyme Poly-Adenosine diphosphate-Ribose Polymerase (PARP).

The DNA of a living cell is constantly exposed to agents and circumstances which may destroy the genomic sequence information of the cell. DNA damage is one of the key factors contributing to cancerogenesis. In this regard, DNA polymerases engaged in DNA replication and repair generate DNA replication errors, thereby introducing potentially disadvantageous mutations. In order to counteract such replication errors, cells activate the cell cycle checkpoint pathway to maintain the integrity of the genome. The cell cycle is stopped or delayed upon detection of DNA damage or unstable DNA replication, thereby allowing repair of damaged DNA sections. Poly-ADP-Ribosylation, or PARylation, is the pivotal modification that is instantly triggered at the DNA damage site, e.g. single-strand breakage, for subsequent DNA damage repair. By PARylation, a residue of ADP-ribose is transferred to target substrates by ADP-ribosyl transferase using NAD⁺. Subsequently, PARP catalyzes the synthesis of poly-ADP-riboses.

PARP proteins contribute to the repair of single-strand breaks (SSBs) in the DNA strand. Without PARP activation, replication of DNA exhibiting SSB(s) is prone to formation of double-strand breakages. PARP inhibition—allowing to suppress that repair mechanism—was shown to be a suitable approach for therapy of various forms of cancer, in particular ovarian, breast, prostate and pancreatic cancer having BRCA1 and BRCA2 mutations.

The PARP inhibitor component of the combination may be provided as a PARP inhibitor monotherapy (one single PARP inhibitor in combination with the radiopharmaceutical as the other component of the combination) or as a combination of distinct PARP inhibitors, e.g. two or more distinct PARP inhibitors.

PARP inhibitors are nicotinamide analogues which competitively bind to the NAD⁺ binding sites of PARP1 and PARP2. PARP inhibitors can be selected from one or more of the group comprising Niraparib, Olaparib, Rucaparib, Talazoparib, Iniparib, Veliparib, Pamiparib, Fluzoparib or Amelparib, including their salts or solvates. Their salts may be alkali or earth alkali salts, if negatively charged at physiological pH conditions. However, typically PARP inhibitors are positively charged at physiological pH conditions. Thus, the anion may be selected from hydrochloride, para-toluenesulfonate, camphorsulfonate, tosylate, hydrobromide, sulphate, phosphate, tannate, oxalate, fumarate, gluconate, alginate, maleate, acetate, citrate, benzoate, succinate, malate, ascorbate, and tartrate. In a preferred embodiment of the invention Niraparib, Talazoparib, Olaparib, Rucaparib or any combination thereof are selected as PARP inhibitor component of the combination. In a more preferred embodiment of the invention, Olaparib and/or Rucaparib, in particular Olaparib or Rucaparib, are used as PARP inhibitor(s) for the combination according to the invention.

Structural formulas of five PARP inhibitors (their respective base form) representing PARP inhibitors to be used for the combination of the present invention are shown below:

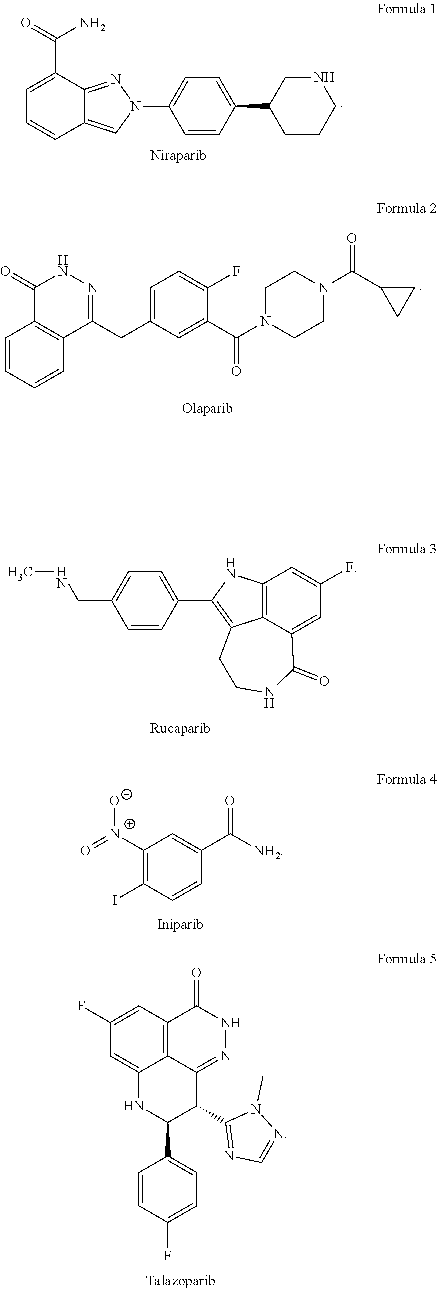

Various of the known and/or approved PARP inhibitors are characterized by a common benzamide core as a pharmacophore, such as olaparib, rucaparib, niraparib, and talazoparib. They differ by their side chains conferring different size and flexibility. They also differ in their PARP trapping efficiencies (talazoparib>niraparib>rucaparib>olaparib). Hence, their half-maximal inhibitory concentrations (IC₅₀) values follow a reverse pattern (talazoparib<niraparib<rucaparib<olaparib). Thus, Talazoparib being about 100 times more potent than olaparib may be used according to one embodiment of the invention, typically at doses being app. 100 times lower than those used for the other above listed PARP inhibitors.

The PARP inhibitor is preferably formulated separately such that it may be administered independently of the administration of the radiopharmaceutical as the other component of the combination of the invention. Its formulation may be solid or liquid. A liquid formulation is required for administration by injection. The PARP inhibitor formulation may thus be formulated for e.g. intravenous administration, e.g. intravenous injection or infusion. Alternatively, the PARP inhibitor may be formulated in solid form, e.g. as a tablet or a capsule. Thereby, the formulation may be suitable for oral administration. The tablet or capsule may contain between 100 and 400 mg PARP inhibitor, unless the PARP inhibitor is talazoparib. The daily dose of talazoparib may be selected from 0.5 to 5 mg, e.g. 0.5 to 2 mg. The formulation of the PARP inhibitor capsule or tablet may be based on matrix polymers. It may comprise one or more of copovidone, silica, mannitol and sodium stearyl fumarate. First, an extrudate may be provided, which is further processed by compression blending. Finally, the PARP tablet may be coated by a standard non-functional film.

Alternatively, the PARP inhibitors may be formulated by nanoparticles, potentially offering superior pharmacokinetics as compared to small molecule drugs. Thereby, the PARP inhibitor may be loaded in specially designed nanoparticles for delivery, in particular for oral administration conferring higher bioavailability. Nanoparticle formulations may overcome off-target drug diffusion issues, rapid elimination and low bioavailability. Nanoparticle formulations may be advantageous in view of the poorly water-soluble characteristics of the free from of PARP inhibitors. Suitable methods for the preparation of PARP inhibitor loaded nanoparticles are the assembly/disassembly method, the nanoprecipitation method, the nano-emulsion method, the hot homogenization method, the solvent evaporation method, and the layer-by-layer method. Advantageous formulations may be nanoparticles, nano-emulsions, nano-capsules, solid lipid particles, lipospheres, and layer-by-layer nanoparticles.

### (C) Further Components of the Combination

The combination according to the invention comprises (i) the radiopharmaceutical and (ii) the PARP inhibitor. One or more distinct PARP inhibitors and/or radiopharmaceuticals may be applied. Two or more PARP inhibitors may be administered commonly or separately by a separate formulation of each of the PARP inhibitors.

In addition, the combination therapy may further comprise one or more other chemotherapy drugs as component (iii). Another such chemotherapy drug may be selected from the group consisting of gemcitabine, temozolomide, cisplatin, cyclophosphamide, carboplatin, paclitaxel, and etoposide. They may be formulated separately or together with the one or more PARP inhibitor(s) for co-delivery. Such a co-formulation may be nanoparticle-based to coordinately release both drugs in vivo.

The combination therapy according to the invention may be combined with vascular endothelial growth factor (VEGF)-targeted agents. By inhibiting VEGF factor (VEGFF) increased DNA damage is observed. Thereby, susceptibility to the effects of PARP inhibition may be increased. In this regard, the combination therapy may be further expanded by administering e.g. at least one VEGF inhibitor selected from the group consisting of sorafenib, sunitinib, nilotinib, pazopanib, dasatinib, cediranib and bevacizumab as a further component (component (iii)).

Immune checkpoint blockade (CPB) may be another target for additional components of the combination therapy according to the invention. PARP inhibitors may be capable of enhancing the efficacy of CPB agents via coordinating activation of robust local and systemic antitumor immune responses. Thus, the combination of the invention may further comprise as component (iii) at least one immune checkpoint inhibitor selected from an anti-PD-1, anti-PD-L1 or anti-CTLA-4 antibody. Such antibodies may be selected from nivolumab, pembrolizumab, durvalumab, atezolizumab, avelumab, ipilimumab, and tremelimumab.

Further, the combination therapy may comprise as component (iii) at least one mTOR inhibitor, e.g. vistusertib, everolimus or sirolimus, or an AKT inhibitor, e.g. capivasertib.

Alternatively, the combination therapy may comprise as component (iii) at least one tyrosine kinase inhibitor as a further anti-cancer drug, e.g. selected from the group consisting of apatinib, vapritinib, capmatinib, pemigatinib, ripretinib, selpercatinib, selumetinib, tucatinib, entrectinib, erdafitinib, fedratinib, pexidartinib, upadacitinib, zanubrutinib, baricitinib, binimetinib, dacomitinib, fostamatinib, gilteritinib, larotrectinib, lorlatinib, acalabrutinib, brigatinib, midostaurin, neratinib, alectinib, cobimetinib, lenvatinib, osimertinib, ceritinib, nintedanib, afatinib, ibrutinib, trametinib, axitinib, bosutinib, cabozantinib, ponatinib, regorafenib, tofacitinib, crizotinib, ruxolitinib, vandetanib, pazopanib, lapatinib, nilotinib, dasatinib, sunitinib, sorafenib, erlotinib, gefitinib, and imatinib.

The combination therapy may also further comprise as component (iii) an inhibitor of an ATR-, ATM-, DNA-Pk-, or Wee-kinase; or, as component (iii) an inhibitor of one or more of AKT, ATM or a DNA-Pk may be applied.

In another embodiment, the combination therapy may comprise at least two additional anti-cancer drugs as component (iii), preferably selected from at least two of the above disclosed groups, e.g. the combination therapy may comprise at least one checkpoint inhibitor and at least one mTOR or at least one check-point inhibitor and at least one tyrosine kinase inhibitor.

Alternatively, the combination therapy may comprise at least two additional anti-cancer drugs from the same group, e.g. may comprise at least two tyrosine kinase inhibitors.

### (D) Tumors, SSTR-Positive Tumors and Neuroendocrine Tumors

The combination (representing the radiopharmaceutical and the PARP inhibitor, respectively) according to the invention is suitable for use in the treatment of cancer and, specifically, SSTR-positive cancer, in particular a solid SSTR-positive cancer. The combination or combination therapy according to the invention may be applied to cancer patients as a first line therapy approach or as a second or third line therapy approach, e.g. following chemotherapy or surgery, e.g. following platinum-based chemotherapy. The SSTR-positive cancer may be an SSTR-2-positive cancer. More specifically, the combination of the invention may be used for the treatment of SSTR-positive neuroendocrine tumors of any organ, in particular the gastro-intestinal tract, the pancreas and the bronco-pulmonary tract. In particular, pulmonary neuroendocrine tumors may be treated by the inventive combination. There are four types of neuroendocrine lung cancers: Small-cell lung cancer (SCLC), large-cell neuroendocrine carcinoma, typical carcinoid and atypical carcinoid, which may be specifically therapeutically addressed.

In a specific embodiment, the therapy approach of the invention is applied to patients which suffer from a primary endocrine neoplasm/tumor and, potentially, metastases thereof. The metastases may be metastases in the lymph nodes or in any other organ, in particular the liver.

In one embodiment of the present invention, the combination can be used for the treatment of any tumor or any neuroendocrine tumor. In a preferred embodiment, the combination is used for the treatment of a neuroendocrine pulmonary tumor, such as small-cell lung cancer (SCLC), large-cell neuroendocrine carcinoma, typical carcinoid and atypical carcinoid. In a more preferred embodiment, the combination is used for the treatment of small-cell lung cancer (SCLC).

The subject to be treated is an animal, in particular a mammalian animal. More specifically, the subject to be treated is a human. The human cancer patient, in particular the cancer patient suffering from a neuroendocrine cancer, may be specifically older than 50 or older than 60, e.g. at the age of between 50 and 70 or 60 to 75. The human cancer patient may be male or female. The human patient may be treated by the combination therapy according to the present invention particularly when being diagnosed with an advanced metastatic setting, e.g. with a late stage neuroendocrine cancer disease, excluding surgery as a first line treatment approach.

For the determination of the extent and size of the cancer, a grading and staging system has been implemented for diagnostic, prognostic and therapeutic purposes. While the cancer grading describes the appearance of cancer cells and tissue, the cancer staging determines how large the primary tumor is and its spreading to other organs in the patient. According to the invention, the combination the therapy may be specifically suitable for the treatment of SSTR-positive cancers or neuroendocrine cancers being staged as GI, GII, GIII or as GIV. These stages define the cancer to have been grown or spread into nearby tissues and potentially into lymph nodes. The higher the stage, the farther the cancer has spread. Alternatively, the combination therapy of the invention may also be applied to a GIV cancer stage with the cancer spread beyond the lymph nodes into other parts of the body. The GIV stage thus typically corresponds to an advanced metastasized stage.

According to the TNM staging mode, the combination therapy according to the invention may be specifically applied to patients being e.g. N1 to N3 staged (cancer spread into the lymph nodes) or e.g. M1 staged (cancer spread to other parts of the body). Thereby, the combination therapy according to the invention may be e.g. used for the treatment of an SSTR-positive cancer patient being staged e.g. as T3, T4 (tumor grading) in combination with e.g. an N2, N3, or M1 according to the TNM system.

To determine the stage of a tumor, several techniques may be employed, such as physical examinations of the patient, molecular imaging techniques comprising X-ray, MRI, CT scans, PET scans and Ultrasound, and further invasive procedures comprising biopsy and surgery.

The cancer patients treated by the inventive combination therapy may be resistant to standard chemotherapy, as exemplified by the frontline treatment based on cisplatin and etoposide. Accordingly, the combination therapy according to the invention may be preferably used for SSTR-positive cancer patients, such as neuroendocrine cancer patients, which have been previously unsuccessfully been treated by a frontline or first line therapy, in particular a combination of cisplatin and etoposide. Such patients may be refractory to frontline treatment.

### (E) Use in a Method of Treatment and Method of Treatment

The combination of the present invention may be used in a method of treating a patient suffering from an SSTR-positive cancer by administering a combination as disclosed herein. Alternatively, the radiopharmaceutical as disclosed herein may be used in a method of treating a patient suffering from an SSTR-positive cancer, whereby the method further comprises the administration of a PARP inhibitor. The specific features of the method directed to using the combination or by combining the use of the radiopharmaceutical and additionally administering a PARP inhibitor correspond to each other. Also disclosed is a method of treating a patient suffering from an SSTR-positive cancer by administering a combination (or a radiopharmaceutical and a PARP inhibitor, respectively) as disclosed herein to the patient. Thereby, the method is directed to the treatment of cancer patients suffering from an SSTR-positive cancer as disclosed herein, such as an SSTR-2 positive cancer. The SSTR-positive cancer may be a neuroendocrine cancer, such as a neuroendocrine cancer of the gastrointestinal tract, of the pancreas or of the broncho-pulmonary tract. In a specific embodiment, it may be a pulmonary neuroendocrine cancer, such as a small-cell lung cancer.

The radiopharmaceutical or the radiopharmaceutical composition, respectively, as component (i) of the combination of the invention may be administered systemically, preferably intravenously, e.g. through infusion drip. The radiopharmaceutical composition may be administered over an extended period of time, e.g. for 10 to 60 min, by infusion, e.g. by a catheter or heparin lock line to be prepared into the vein of a subject, and may be flush with the appropriate saline and or heparin solution. The dose may be administered via luer-lock into the catheter or heparin lock line. The site of administration may depend on the patient's tumor/metastases profile. One specific site of administration may the arm vein. Preferred administration routes of the radiopharmaceutical or the radiopharmaceutical composition, respectively, are intravenous or intratumoral administration.

The radiopharmaceutical as described herein may be administered only once (single administration) or only once per treatment cycle. In particular, the radiopharmaceutical as component (i) of the combination is preferably administered not more than seven times, more preferably not more than five times, even more preferably not more than 3 times. Advantageously a single or very few administrations of the radiopharmaceutical is/are sufficient to exert its anti-tumor effects.

The PARP inhibitor component may be administered by the oral or the intravenous route. Alternatively, the PARP inhibitor may be administered intratumorally. Oral administration may be preferred. The dosing of the PARP inhibitor for the combination therapy may vary depending on the tumor to be treated, the patient (e.g. its weight, sex etc.), the specific PARP inhibitor administered and other factors. It may be administered by a once or twice daily dosage regimen in the course of one or more PARP inhibitor “treatment cycles”. The total dosage per day may be in the range of 100 mg and 800 mg depending on the PARP inhibitor used, such as between 200 mg and 600 mg. The total daily dosage may be administered by 1 to 4 or 2 to 4 dosage units. For the PARP inhibitor talazoparib the daily dosage to be administered (e.g. once or twice daily) may be in the range of 0.5 mg to 5 mg or at 0.5 to 1.5 mg, e.g. at 1 mg. For the PARP inhibitor olaparib, the daily doses (e.g. administered once or twice daily) may be at 600 to 1200 mg, e.g. at 400 mg twice daily. For the PARP inhibitor niraparib, the daily doses (e.g. administered once or twice daily) may be at 200 to 500 mg, e.g. at 300 mg daily. For the PARP inhibitor Rucaparib, the daily doses (e.g. administered once or twice daily) may be at 800 to 1500 mg, e.g. at 600 mg twice daily.

In an embodiment of the method of the present invention, component (i) is administered intravenously and component (ii) is administered orally or intravenously, preferably orally.

The PARP inhibitor may be administered over an extended period of time of preferably more than 5 days, e.g. 5 to 15 days or e.g. 5 to 30 days, e.g. on a daily basis for 5 to 30 or 5 to 15 days, e.g. 5 to 10 days per “treatment cycle”. An uninterrupted period of daily (or whatever other treatment regimen, e.g. every second day or every third day) administration of the PARP inhibitor is defined as a “treatment cycle”. The components of the combination according to the invention may be subject to one uninterrupted “treatment cycle” or repeated “treatment cycles, e.g. 2 or more “treatment cycles”, such as 2 to 10 “treatment cycles”. One single uninterrupted “treatment cycle” applied until the end of the therapy may be considered as a continuous treatment. The “treatment cycles” may be interrupted, e.g. in case of adverse reaction, preferably by a period of 1 to 15 days, e.g. 2 to 8 days. Preferably, the treatment by a PARP inhibitor (Lynparza®, Rubraca®, Zejula®) may be carried out according to the treatment protocols as approved for each of these PARP inhibitors by the Federal Drug Administration (FDA) (www.fda.gov) or by the European Medicines Agency (EMA) (www.ema.europa.eu).

The administration protocol of the combination may be based on a concurrent or an intermittent approach.

In one embodiment, the concurrent regimen allows to administer both components in parallel. When using the concurrent approach, the radiopharmaceutical may be administered in the course of the first treatment cycle or any following treatment cycle with the PARP inhibitor. Advantageously, the radiopharmaceutical is administered only once per treatment cycle. It may even administered only once over the course of the entire treatment characterized by a series of treatment cycles with the PARP inhibitor. If administered only once in the course of the treatment at all, the radiopharmaceutical may be preferably administered in the course of the first or the second treatment cycle, more preferably in the course of the first treatment cycle. If administered twice or more in the course of the entire treatment, the initial administration will be preferably carried out in the first or the second treatment cycle as well. The administration day of the radiopharmaceutical depends on the length of the treatment cycle. Typically, it will be no earlier than at day 3 of the treatment cycle and no later 3 days prior to the termination of the first treatment cycle. Advantageously, the radiopharmaceutical will be administered at no earlier than at about ⅓ of the treatment cycle period and/or no later than about ⅔ of the treatment cycle period. Whether the radiopharmaceutical is repeatedly administered in the over the series of treatment cycles with the PARP inhibitor may depend on the patient's individual cancer disease. If repeated administration of the radiopharmaceutical is envisaged, repeated administration at e.g. cycle 2 and/or cycle 3 etc. may follow the administration scheme described above.

Concurrent administration as e.g. exemplified above means that the radiopharmaceutical and the PARP inhibitor are administered at least once on the same day, e.g. in the course of the first treatment cycle. Administration “on the same day” may mean administration at “about the same time” or at “different times”, e.g. in the morning and the evening.

“At about the same time”, as used herein, refers in particular to simultaneous administration. It also encompasses situations, where directly after administration of the radiopharmaceutical the PARP inhibitor is administered or directly after administration of the PARP inhibitor the radiopharmaceutical is administered. The skilled person understands that “directly after” includes the time necessary to prepare the second administration—in particular the time necessary for exposing and e.g. disinfecting the site for the second administration as well as appropriate preparation of the “administration device” (e.g., syringe, pump, etc.). At about the same time” includes, as a matter simultaneous administration as well, e.g. simultaneous administration by the PARP inhibitor (e.g. oral or intravenous) and the radiopharmaceutical (e.g. intravenous).

In another embodiment, components (i) and (ii) are administered intermittently. When administering intermittently, component (ii) may be initially administered by a first treatment of cycle of e.g. 1 to 15 days, preferably 5 to 15 or 5 to 10 days. Upon termination of the first treatment cycle with the PARP inhibitor, the radiopharmaceutical may be administered prior to the onset of the second treatment cycle. It may be advantageous to administer the radiopharmaceutical with a delay of at least 1 day, preferably 1 to 3 or 1 to 5 days upon termination of the first treatment cycle with PARP inhibitor. Preferably, at least one day, more preferably 1 to 3 days or 1 to 5 days after the administration of the radiopharmaceutical, a second treatment cycle with the PARP inhibitor may be started. The number of PARP inhibitor treatment cycles intermittent with the administration of the radiopharmaceutical may be 1 to 4 or 3 to 6 or more depending e.g. on the patient's tumor. As described above, the radiopharmaceutical may be administered only once or by number which is smaller than the number of interruptions between the treatment cycles with the PARP inhibitor. Thus, the radiopharmaceutical may e.g. be administered initially only after termination of the second treatment cycle and prior to the third treatment cycle or at a later stage of the treatment.

The inventive combination based on components (i) and (ii) may be administered according to the following basic treatment schedule: In one embodiment, component (ii) is continuously administered over the entire treatment period. According to that embodiment, medication of component (ii) is not discontinued when the radiopharmaceutical is administered. In another embodiment, component (ii) is continuously administered. However, the medication by component (ii) is interrupted for an interval or a time window defined by the administration of component (i). The interval characterizing interruption of administration of component (ii) may start e.g. 1 to 12 days before and end 1 to 12 days after administration of component (i). A still further embodiment of a treatment schedule of the inventive combination is based on discontinuous administration of component (ii). Thereby, component (ii) is exclusively administered within an interval or a time window defined by the administration of component (i), e.g. starting 1 to 12 or 5 to 12 days before and ending 1 to 12 or 5 to 12 days after administration of component (i).

Component (ii) is preferably administered based on daily uptake within its administration period(s). A dosage range of 50 to 800 mg as a total daily dosage within the treatment period during which component (ii) is applied, may be preferred. Daily administration may either represent a once or, preferably, a twice daily administration regimen. Thereby, e.g. 25 mg to 50 mg may be administered once or twice daily, 50 mg to 100 mg may be administered once or twice daily, 150 mg to 200 mg may be administered once or twice daily, or 250 mg to 400 mg of component (ii) may be administered once or twice daily.

While component (ii) may be applied continuously for the entire treatment period of the combination as described above, discontinuous administration of component (ii) aligning medication of both components may be another option for the basic treatment schedule of the inventive combination. Administration of component (ii) may be started prior to each radiotherapy cycle by component (i). Thereby, component (ii) may be administered for less than 4 weeks, 3 weeks, 2 weeks, or for less than 1 week prior to and/or after each radiotherapy cycle by component (i), in particular for 2 to 12 days prior to and/or after administration of component (i), preferably prior to and after administration of component (i). The administration schedule of component (ii) prior to and after administration of component (i) is not necessarily symmetric. Thus, the period of administering component (ii) prior to the administration of component (i) does not need to be same as the subsequent period after administration of component (i). Thereby, component (ii) may be administered e.g. for 2 weeks prior to the administration of component (i) and for only 1 week after the administration of component (i) or vice versa. Thus, the medication period of component (ii) preceding administration of component (i), in case of an discontinuous administration schedule of component (ii), may be longer than the medication period of component (ii) following administration of component (i) or vice versa. The difference may e.g. be 1 to 3 weeks.

In case of an extended interval between at least two radiotherapy treatment cycles by component (i), discontinuous administration of component (ii) may extend as well to e.g. 4 to 12 weeks prior to and/or after each treatment cycle or for 4 to 8 weeks, 6 to 10 weeks or 8 to 12 weeks prior to and/or after each radiopharmaceutical therapy cycle.

Component (i) of the inventive combination is typically administered every 4 to every 12 weeks, e.g. every 4 to 6 weeks or every 6 to 8 weeks or every 8 to 10 weeks or every 10 to 12 weeks. The frequency of the treatments of component (i) and the number of treatments with component (i) may depend on the tumor to be treated, the dosage of the radiopharmaceutical applied by the treatment cycles, the patient's overall health status etc. Under standard medication conditions, component (i) may be applied 3 to 10 times. The total treatment period involving component (i) is thus defined by the number and the frequency of single treatments of component (i).

Component (i) is typically applied in a clinical setting allowing for collection of the radiopharmaceutical excreted by the patient after its administration. Component (i) is advantageously applied as an injection solution, e.g. by intravenous infusion. The administration may last for 1 to 10 h or 1 to 6 h. The dosage of the radiopharmaceutical as component (i) may be advantageously selected such that 2 to 9 GBq or 3 to 5 GBq, 2 to 7 Gbq, 4 to 7 GBq, 5 to 9 GBq, or 7 to 9 GBq are applied per treatment cycle. The dosage of the radiopharmaceutical may be administered based on the patient's body weight such that e.g. 40 MBq/kg to 160 MBq/kg, preferably 60 MBq/kg to 140 MBq/kg, most preferably 80 MBq/kg to 120 MBq/kg are applied per radiotherapy treatment cycle. The concentration of component (i) in the formulation to be administered per treatment cycle may be 250 MBq/mL to 750 MBq/mL, preferably 330 MBq/mL to 520 MBq/mL, preferably 375 MBq/mL to 470 MBq/mL, most preferably 400 MBq/mL to 440 MBq/mL.

The total treatment period including all treatment cycles defined by the administration of component (i) may range from 25 weeks to 100 weeks. In some embodiments of the treatment schedule of the inventive combination, component (ii) may be administered continuously for up to 30 weeks, up to 12 months, or up to 24 months, optionally starting prior to the first administration of component (i) (first radiotherapy treatment cycle), e.g. 1 to 6 weeks prior to the first treatment cycle, and/or ending only after the ultimate administration of component (i) (ultimate radiotherapy treatment cycle), e.g. 1 to 6 weeks after the ultimate treatment cycle.

The treatment schedule may be based on a dose escalation scheme. Thereby, the dosage of at least one of the components of the inventive combination may be increased as a function of time. E.g. the dosage of component (i) may be increased by 10 to 50%, preferably 10 to 25% from one treatment cycle to the next treatment cycle. Alternatively, the escalation may be based on a consistent increase (from one cycle to the next cycle) of radioactivity by a dosage value within the range of from 0.5 to 2 GBq. Such a dosage escalation scheme may be also applied for one or more of the following treatment cycles as well, depending e.g. on potential adverse reactions by the patient to the dosage increase and/or depending on the patient's tumor load. Alternatively or additionally, the dosage of component (ii) may be escalated in the course of the treatment period, e.g. by an increase of 10 to 50%, preferably 10 to 25%. Alternatively, dose escalation of component (ii) may be based on a consistent increase of the dosage by a dosage within the range of from 10 mg to 100 mg, e.g. 50 mg.

The escalation may be applied in alignment with the escalation of the dosage of component (i) or independently thereof. The dosage of component (ii) may be escalated every 4 to 10 weeks, e.g. every 5 to 7 weeks.

### (F) Combined Diagnostic and Treatment Steps

The treatment of an SSTR-positive cancer patient by the combination of the invention may be preceded by a diagnostic or detection step, which may be an in vitro approach (e.g. by immune histological staining) or, in particular, in vivo diagnostics for locating the patient's tumor cells in its body. Thereby, the combination may be used in a method combining diagnosis and treatment. Selective receptor-targeting radiopeptides have emerged as important class of radiopharmaceuticals for molecular imaging and therapy of tumors that overexpress peptide receptors, such as SSTRs. Thus, the radiopharmaceutical comprising the SSTR binding compound may be labeled with gamma-emitting radionuclides. Upon administration of the radiopharmaceutical, the tumor or tumor sites may be identified e.g. by single photon emission computed tomography (SPECT) or positron emission spectroscopy (PET). Thus, the diagnostic step allows for the selection of patients which express the target receptor on their cancer cells, e.g. a somatostatin receptor. The same (or another) radiopharmaceutical labeled with a beta-particle emitting radionuclide (damaging the cancer cells in a targeted manner) instead of e.g. a gamma-emitting radionuclide may be applied according to the combination therapy of the present invention in a subsequent cancer treatment step.

For diagnostic purposes by e.g. SPECT or SPECT/CT, the radioisotope ¹⁸F may be used. When preferably employing a metal radioisotope, the radiopharmaceutical described above may be labelled with a radionuclide selected from the group consisting of ^{99m}Tc, ¹²³I, ¹¹¹In and ¹⁵⁵Tb. In a more preferred embodiment, the radionuclides are selected from the group consisting of ¹¹¹In and ¹⁵⁵Tb.

Alternatively, the in vivo diagnostic step may be carried out by positron emission tomography (PET) carried out in combination with CT or MRT. By PET, two gamma photons are detected upon decay of a positron emitting radionuclide. When applying PET, the radionuclide may be ¹¹C, ¹³N, ¹⁵O or ¹⁸F. When applying metal radioisotopes being coordinated by a chelator, they are preferably selected from the group consisting of ¹¹⁰In, ^{94m}Tc, ⁸⁶Y, ⁶⁰Cu, ⁶¹Cu, ⁶⁴Cu, ⁶⁶Ga, ⁶⁸Ga and ⁸²Tb. In more preferred embodiment, the radionuclides are selected from the group consisting of ⁶⁸Ga, ⁶⁶Ga, ⁶⁰Cu, ⁶¹Cu, ⁶⁴Cu and ⁸²Tb.

In addition or alternatively, the initial detection step may include a step of screening for a gene mutation, e.g. by next generation sequencing or appropriate PCR protocols. The gene mutation may preferably be selected from the group consisting of a DNA repair gene mutation, more specifically a gene mutation involving homologous recombination repair (HRR), a BRCA1/2, ATM, BARD1, BRIP1, CDK12, CHEK2, FANCL, PALB2, RAD51B, RAD51C, RAD51D, and a RAD54 mutation.

Once the SSTR-positive cancer, e.g. the neuroendocrine cancer, has been diagnosed and visualized by the above molecular imaging techniques, cancer treatment may be carried out by a radiopharmaceutical as disclosed herein. Thus, the radiopharmaceutical used for the initial detection step may—by an embodiment—be identical to the radiopharmaceutical used for the treatment step in terms of the chelator and the Somatostatin receptor binding compound, but different in terms of the radionuclide (being typically a 9 particle emitter).

By the combined approach of initially detecting an SSTR-positive cancer, preferably in vivo, and thereafter treating the patient by the inventive combination comprising a radiopharmaceutical and a PARP inhibitor diagnosis and therapy of an SSTR-positive cancer is enabled, in particular of a neuroendocrine cancer, a neuroendocrine pulmonary cancer and specifically a small-cell lung cancer.

In addition, the therapy progress may be monitored by PET/CT or SPECT/CT scans in the course of the therapy, e.g. after each treatment cycle. SPECT may preferably serve as a technology to study the distribution of the radiopharmaceutical in the body. Moreover, imaging techniques may be used to monitor the tumor volume, e.g. tumor shrinkage, in the course of the therapy.

### (G) Kit or Kit of Parts

Another aspect of the present invention refers to a kit or kit of parts comprising the combination as disclosed herein. Typically, the two components of the combination are provided as two distinct entities, which may be represented by two or more parts of the kit. One or both of the two entities may be provided as “ready to use” formulation(s) or as a pre-formulated components. If both components are provided as “ready to use” formulations, they may be represented by two part of the kit. If at least one of the components is not provided as a “ready to use” formulation, the kit may have more than two, e.g. 3 or 4 parts. In case of their use for oral administration, in particular of the PARP component, they will typically be provided in a “ready to use” format. In case of their use by injection or infusion for e.g. intravenous administration, the components may be provided in a pre-formulated form requiring the final preparation “on site”. Such a scenario may typically apply to the radiopharmaceutical or the radiopharmaceutical composition, respectively.

Thereby, a labelling reaction with the radionuclide may be required to be carried out in a clinical hospital or laboratory setting for the provision of the “ready to use” radiopharmaceutical (composition) component of the combination of the invention. In such instances, the various reaction ingredients may be provided to the user in the form of parts of a “kit”. Accordingly, a kit of the invention may be adapted for preparing a radiopharmaceutical composition. It may comprise (1) a somatostatin receptor (SSTR) (ant)agonist as described herein conjugated to the radionuclide's chelator, (2) an inert pharmaceutically acceptable carrier and/or formulating agent with optional adjuvants, (3) a solution of a radioactive metal isotope, typically in the form of its salt, and, optionally, (4) instructions for use with a prescription for reacting the ingredients present in the kit, all of the above as parts of a kit. Components (1), (2), (3) and, optionally, (4) are typically provided as separate parts by the kit.

Alternatively, the components may be provided as a combination of two distinct kits. Thus, component (i) may be provided as a first kit or kit of parts and component (ii) as a second kit or kit of parts. It may be preferred to combine a first kit of parts comprising component (i), the radiopharmaceutical as described above, and a second kit or kit of parts comprising component (ii), the PARP inhibitor. The first and the second kit (of parts) may thus be delivered as separate entities to the site of use allowing “right on time” provision of the radionuclide for its further formulation and subsequent therapeutic usage.

The somatostatin antagonist (peptidic component (c) of the radiopharmaceutical) may be conjugated by a reaction with a chelating agent (component (b) of the radiopharmaceutical) as defined hereinbefore. The resulting peptide/chelator conjugate provides an advantageous entity for stably complexing the radioisotope of the radiopharmaceutical) in a straight-forward manner at the site of use. The peptide/chelator conjugate, e.g. in the form of its salt, may be provided in dry form or, more typically, in solution as part of a kit, e.g. in a buffered or non-buffered aqueous solution. When being provided in dry form, it may be delivered in lyophilized form, such that the lyophilized conjugate has to be dissolved in solution at the site of use. Due to its character as an injection liquid, it should be sterile. When the constituent is in the dry state, the user should preferably use a sterile physiological saline solution as a solvent, which is optionally buffered.

The (a) radionuclide/-isotope or its salt as a component of the kit or kit of parts kit is typically provided in an aqueous acidic solution, e.g. in a hydrochloric acid solution, exhibiting a pH of e.g. <2 or <1. As the radionuclide is provided as a separate part of the kit, the radionuclide has to be prepared for binding to the peptide/chelator conjugate by a complex-forming reaction. Advantageously, both the solution containing the peptide/chelator conjugate and the solution containing the radionuclide are combined. The solution containing the peptide/chelator conjugate may advantageously contain a buffer buffering the pH in the acidic range, e.g. acetate, allowing the complexation reaction to occur under appropriate acidic conditions. For preventing precipitation of the radioactive metal (e.g. by the formation of its hydroxy salt), the complexation reaction should not proceed under alkaline conditions. Advantageously, the complexation reaction is carried under conditions shifting the equilibrium towards complexation, e.g. by heating, e.g. to a temperature close to the solution's boiling point, the combined solutions of the radionuclide and the peptide/chelator.

In addition, the resulting product may be finally re-formulated by adding stabilization additives as component (2) of the kit. Thereby, the radiopharmaceutical may be stabilized with suitable stabilizers, for example, ethanol, ascorbic acid, gentisic acid or salts of these acids.

The kit may be a kit of two or more parts comprising any of the components exemplified above in suitable containers. For example, each container may be in the form of vials, bottles, squeeze bottles, jars, sealed sleeves, envelopes or pouches, tubes or blister packages or any other suitable form, provided the container preferably prevents premature mixing of components. Each of the different components may be provided separately, or some of the different components may be provided together (i.e. in the same container), as described above.

A container may also be a compartment or a chamber within a vial, a tube, a jar, or an envelope, or a sleeve, or a blister package or a bottle, provided that the contents of one compartment are not able to associate physically with the contents of another compartment prior to their deliberate mixing by a pharmacist or physician

### EXAMPLES

The following experiments demonstrate the increased cell killing effect of ¹⁷⁷Lu-DOTATOC in combination with PARP inhibitors and biodistribution of ⁶⁸Ga-DOTATOC in various organs.

### Example 1

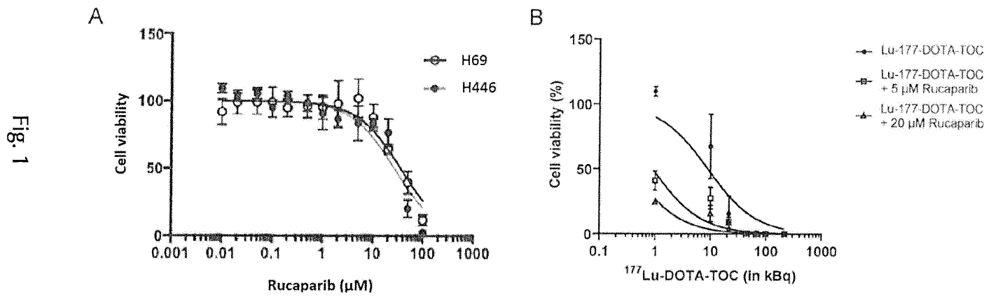

Viability of two different human SCLC cell lines (H446 and H69J, expressing the SSTR2, was studied in an IC₅₀ assay upon addition of three different PARP inhibitors (Rucaparib, Olaparib, and Talazoparib}. DNA double strand breaks were mapped by histone yH2AX fluorescence microscopy. The results show a significantly increased cell killing effect in H446 and H69 cell lines whenever a PARP inhibitor was combined with ¹⁷⁷Lu-DOTATOC. Both cell lines express relatively low levels of SSTR2 (compared with the standard AR42J cell line). Among them, H69 express higher SSTR2 levels than H446. Cell viability was found to be significantly decreased in “low” (H446) and “high” (H69) SSTR2 expressing cell lines when PARP inhibitors were combined with ¹⁷⁷Lu-DOTATOC (cf. FIG. 1A). Cell viability was slightly reduced in both SCLC cell lines upon addition of 20 μM of Rucaparib alone. That effect was not observed at lower concentrations (IC₅₀ H69: 36 μM, IC₅₀ H446: 32 μM). Combined addition of Rucaparib and ¹⁷⁷Lu-DOTATOC according to the invention led to a pronounced decline of cell viability in H446 cells, even at very low activity concentrations of 1 and 10 kBq/well. 50% of all cells (IC₅₀) were kept alive at a dosage of 8.9 kBq of ¹⁷⁷Lu-DOTATOC. The IC₅₀ value was significantly reduced to 0, 4 kBq only, upon addition when ¹⁷⁷LU-DOTATOC was combined with 20 μM of Rucaparib and 0.9 kBq in combination with 5 μM Rucaparib. At such concentrations, neither Rucaparib nor ¹⁷⁷Lu-DOTATOC, respectively, alone have any major impact on cell viability. The activity required for ¹⁷⁷Lu-DOTATOC to reduce cell viability to 50% by H446 cells in the combination treatment was reduced by a factor of 10 (5 μM Rucaparib) to 22 (20 μM Rucaparib).

It was thus demonstrated that synergistic effects are induced when applying the combination treatment according to the invention.

Further evidence for such a synergistic effect was collected by analogous viability assays employing other PARP inhibitors, such as Olaparib and Talazoparib, in combination with ¹⁷⁷Lu-DOTATOC. The applied PARP inhibitor concentrations for combination therapy was adjusted depending on their individual cytotoxicity. 5 μM of Olaparib and 10 nM of Talazoparib were added to the cell cultures in combination with various doses of ¹⁷⁷Lu-DOTATOC. As observed in the experiments with Rucaparib as PARP inhibitor, the IC₅₀ values of ¹⁷⁷Lu-DOTATOC were significantly reduced when combined with the other PARP inhibitors as well. That finding was proven in all cell lines.

All of the experiments support the conclusion that a synergistic effect is observed, regardless of the cell lines employed for the viability assay and regardless of the PARP inhibitor added. H446 cells were found to consistently exhibit more pronounced synergistic effects than H69 cells. That finding is in line with previous reports on H69 cells' lower sensitivity to Peptide Receptor Radionuclide Therapy (PRRT) despite their higher SSTR2 expression levels. It is noted that at lower activity doses (1-10 kBq/well) of the radiopharmaceutical, combination treatment with PARP inhibitors has a strong effect on cell viability when comparing combination treatment with individual treatment of either of the radiopharmaceutical or the PARP inhibitor alone.

### Example 2

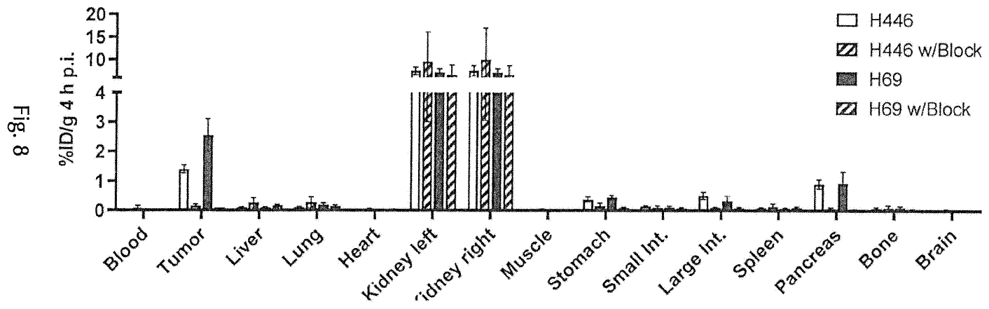

Tumor accumulation of ⁶⁸Ga-DOTATOC (10 MBq) was evaluated in SCLC xenograft mouse model by subcutaneous inoculation of 2-3 mio SSTR2 expressing H446 and H69 cells, respectively, into athymic nude mice. The results (obtained from PET scans) shown in FIG. 8 reveal a visible uptake of ⁶⁸Ga-DOTATOC in the tumor after 4 hours p.i. The accumulation was found to be in line with the known expression levels of the two used SCLC cell lines, with values of around 1-3% ID/g. Increased uptake was found in H69 cells, which exhibits a higher expression rate of SSTR2 than H446 cells. Significantly higher uptake is usually observed in rat pancreatic AR42J (8-12% ID/g at 4 hours p.i. at similar conditions, which represents a widely established animal model for radiopharmaceutical SSTR2 treatment. Increased efficacy of the combination treatment and ⁶⁸Ga-DOTATOC monotherapy in H69 SCLC cell lines (vs. H446 SCLC cell lines) was concluded to result from increased uptake of ⁶⁸Ga-DOTATOC in H69 cells. Uptake in H69 cells was found to be app. 2-fold higher than in H446 cells. The results may be considered as being representative for sub-groups of SCLC patients expressing more (corresponding to the results with H69 cells) or less (corresponding to the results with H446 cells) SSTR2 on their cancer cells.

### Example 3

Tumor bearing animals were studied by administration of ⁶⁸Ga-DOTATOC. Small animal positron emission tomography revealed the presence of the SSTR2 in the course of the whole treatment period. The expression status of SSTR2 did not vary over time, in particular it did not decline in the course of the treatment. The image quality corresponded to the image quality reported for human patients underlining the importance of PET imaging for potential patient selection for envisage treatment of a neuroendocrine tumor, such as SCLC.

### Example 4

Example 4 presents results of comparative in vivo studies based on two distinct xenograft mice models (based on H446 and H69 SCLC cells, respectively) involving PARP inhibitors Rucaparib (administered to both xenograft mouse models) and Olaparib (administered to the H69 xenograft mouse model).

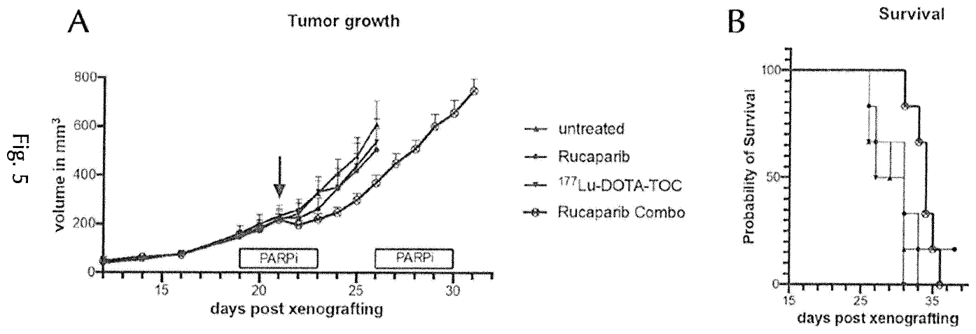

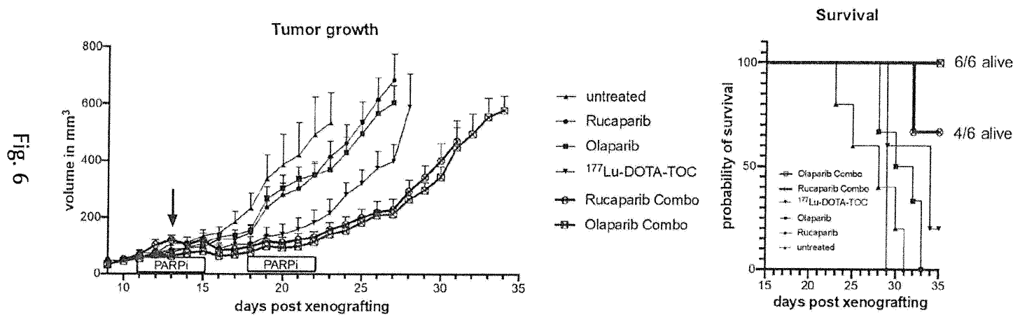

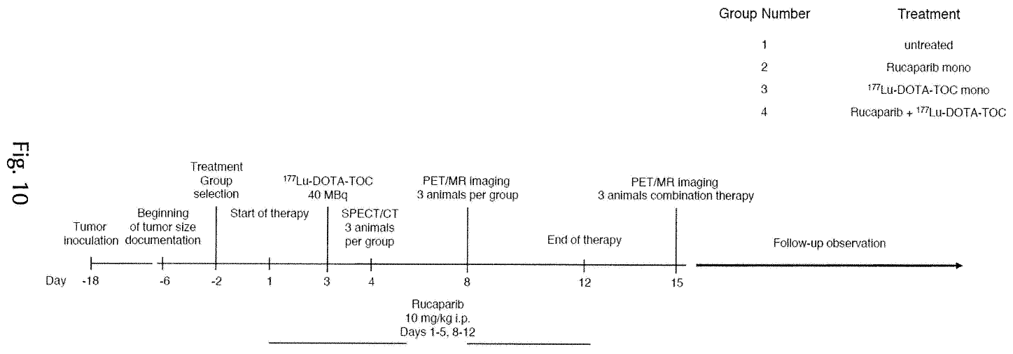

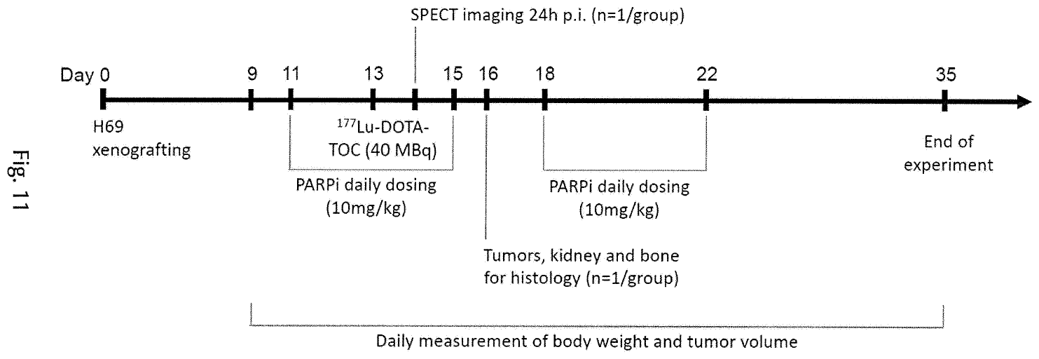

The results of the cell viability experiments were transferred to an in vivo model. H446 (FIG. 5) or H69 (FIG. 6) SCLC cells were subcutaneously inoculated in athymic nude mice to establish xenograft mouse models (injection of about 2 mio. cells in 200 μl Medium/Matrigel (1:1). Tumor volume and body weight were monitored daily during the study period. The differences of the initial tumor volume and the body weight of the mice of each treatment arm were negligible. The animals were treated with either PARP inhibitor alone or ¹⁷⁷Lu-DOTATOC alone, or with a combination of PARP inhibitor and of ¹⁷⁷Lu-DOTATOC. The onset of the treatment was at day 19 (H446 model) or at day 11 (H69 model) (administration of the PARP inhibitor), respectively. These groups were compared to an untreated control group. While the radiopharmaceutical was administered to the animals only once, the animals were treated with PARP inhibitor in two cycles of five days each (10 mg/kg in 200 μl PBS i.p.) at days 19 to 23 and 26 to 30 or at days 11 to 15 and 18 to 22, respectively. The treatment was interrupted for two days after the end of the first PARP inhibitor treatment cycle. ¹⁷⁷Lu-DOTATOC (40 MBq, 223 MBq/nmol) was administered in the middle of the first PARP inhibitor treatment cycle (at day 21 or at day 13, respectively). Study endpoint was a tumor size exceeding 15 mm in each dimension. The results of tumor growth and survival are shown in FIG. 5 (see trial protocol according to FIG. 10 based on H446 cell xenograft model)) and FIG. 6 (see trial protocol in FIG. 11 based on a H69 cell xenograft model), respectively. Monotherapy arms based on the radiopharmaceutical or on the PARP inhibitor, respectively, alone showed no significant delay of tumor growth in H446 xenografts (SSTR2 low-expressing SCLC) versus the untreated control group (FIG. 5). There was also no significant effect observed in overall survival for monotherapy treatment arms vs. control. Combination therapy (FIG. 5) was e.g. found to lead to a significantly longer survival than treatment with ¹⁷⁷Lu-DOTATOC (p<0.01). 50% of all animals died or needed to be euthanized between day 25 and day 30 upon onset of the experiment.

Combination treatment according to the invention based on Rucaparib and ¹⁷⁷Lu-DOTATOC was found to be characterized by a significant improvement of the overall survival in the H446 animal model (FIG. 5). 50% of all animals were euthanized on day 34 only. Tumor growth was significantly delayed in the groups treated by the inventive combination approach. The animal ultimately reaching the terminal tumor size was euthanized on day 36 only.

The findings in H446 cell lines were confirmed in the H69 cell based animal model (FIG. 6), with H69 cells expressing SSTR2 more strongly. By that experimental set-up, 4 out of 6 or 6 out of 6 animals survived following combination therapy, while monotherapy by the PARP inhibitors did not rescue any of the 6 animals treated. 1 out of 5 animals (1 animal excluded from the study) survived upon treatment with ¹⁷⁷Lu-DOTATOC monotherapy.

By the experimental set-up based on the H69 cell based animal model, an additional trial arm was studied based on administration of Olaparib instead of Rucaparib as the PARP inhibitor. In conformity with the results observed for tumor growth delay data in the H446 cell based animal model (FIG. 5), single therapy based on the PARP inhibitor alone did not result in major anti-cancer effects, as expected since tumor cell lines H446 and H69 lack BRCA1/2 mutations. No significant therapeutic difference between Olaparib and Rucaparib was observed by that experimental set-up. Thus, the advantageous effects of the combination therapy are observed irrespective of which PARP inhibitor is administered.

Monotherapy treatment with ¹⁷⁷Lu-DOTATOC for the H69 cell based animal model (FIG. 6) in terms of delay in tumor growth and increased survival deviates from the results observed from the H446 experiments (FIG. 5). That finding may be due to the increased expression rate of SSTR2 in H69 cells (cf. FIG. 8). Without being bound to any theory, higher radiation damage may be induced—due to the higher SSTR2 expression rate—by the elevated tumor uptake of the radiopharmaceutical. The response to the combination of ¹⁷⁷Lu-DOTATOC with either Rucaparib or Olaparib exhibited a significant tumor growth delay. At the termination end point 35 days after the onset, animals in both groups were still alive.

As a result, dual combination therapy according to the invention, using the combination of a radiopharmaceutical and a PARP inhibitor, generate superior effects as compared to monotherapies, including ¹⁷⁷Lu-DOTATOC monotherapy. The combination therapy was generally well tolerated by the animals. Body weight monitoring data were comparable in all treatment groups versus the untreated control group. The applied doses of PARP inhibitor were rather low. Its radio sensitizing effect results in a significant response to radioligand therapy even at significantly lower doses—as established by the experimental data.

### Example 5

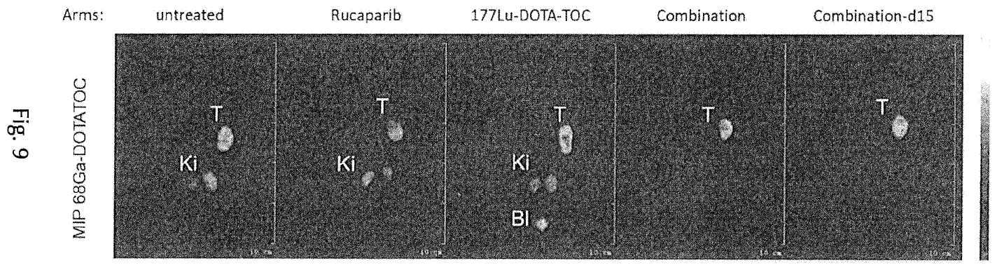

In vivo imaging studies in the SLCL mouse model were carried out. The mouse model is based on H4456 tumor cell bearing mice. They were studied by small animal PET using ⁶⁸Ga-DOTATOC. The presence of the SSTR receptor, here SSTR-2, was analyzed in the course of the treatment protocol. The SSTR-2 expression status did not decline or alter in the course of the treatment. The results are shown in FIG. 9. It may be concluded that that PET imaging may be beneficial to select the appropriate patient cohort, e.g. SCLC patients.

### CONCLUSIONS

By the present invention, a combination of two components comprising a radiopharmaceutical as the first component and a PARP inhibitor as the second component was shown to efficiently combat SSTR-positive cancer cells by experimental in vitro and in vivo settings. By the inventive approach tumor cell viability is significantly and synergistically reduced as compared to treatment protocols based on either of the two components alone. Moreover, the tumor growth is significantly delayed when treating the subjects by the combinatory approach according to the present invention (as compared to the treatment by either of the two components alone). Thus, the combination therapy is considered to achieve long-lasting tumor remission and stabilization.

1. A combination comprising:
(i) a radiopharmaceutical comprising (a) a radionuclide, (b) a chelator, and (c) a Somatostatin receptor binding compound; and
(ii) a PARP inhibitor.

2. The combination according to claim 1, wherein the radionuclide is selected from a group consisting of ¹⁷⁷Lu, ⁹⁰, ⁶⁴Cu, ¹⁵⁵Tb, ¹⁶¹Tb, ¹⁸⁸Re, ²²⁵Ac, ²¹³Bi, ^{99m}Tc, ¹²³I, ¹¹¹In, ⁶⁸Ga, ⁶⁶Ga, ⁶⁰Cu, ⁶¹Cu, and ⁶⁴Cu.

3. The combination according to claim 1 or 2, wherein the radionuclide is selected from a trivalent, R radiation emitting radionuclide.

4. The combination according to any of claims 1 to 3, wherein the radionuclide is selected from ¹⁷⁷Lu, and ⁹⁰Y.

5. The combination according to any of claims 1 to 4, wherein the chelator is a macrocyclic chelator.

6. The combination according to claim 5, wherein the chelator is selected from the group consisting of DOTA, NOTA, NODAGA, HBED, HBED-CC, DOTAM, TRAP, NOPO, PCTA and EDTA and derivatives thereof.

7. The combination according to any of claims 1 to 6, wherein the Somatostatin receptor binding compound is a Somatostatin receptor agonist or antagonist.

8. The combination according to claim 7, wherein the Somatostatin receptor agonist or antagonist is a peptide or a peptide analog, preferably a cyclic peptide or peptide analog.

9. The combination according to claim 8, wherein the Somatostatin receptor agonist is selected from the group consisting of TOC, TATE or NOC or wherein the Somatostatin receptor antagonist is JR11 or LM3, in particular TOC.

10. The combination according to any of claims 1 to 9, wherein component (ii) is ¹⁷⁷Lu-DOTATOC.

11. The combination according to any of claims 1 to 10, wherein the (ii) PARP inhibitor is selected from the group consisting of Niraparib, Olaparib, Rucaparib, Talazoparib, Iniparib, Veliparib, Pamiparib; Fluzoparib, and Amelparib or any combination thereof.

12. The combination according to claim 11, wherein the (ii) PARP inhibitor is selected from the group consisting of Rucaparib, Olaparib, Niraparib and Talazoparib or any combination thereof.

13. The combination according to any of claims 1 to 12, wherein the combination comprises two or more distinct PARP inhibitors, preferably two distinct PARP inhibitors.

14. The combination according to any of claims 1 to 13, wherein the (i) radiopharmaceutical and (ii) PARP inhibitor are formulated separately.

15. The combination according to claim 14, wherein the (i) radiopharmaceutical is formulated for intravenous administration and the (ii) PARP inhibitor is formulated for oral or intravenous administration.

16. The combination according to any of claims 1 to 15, wherein the combination further comprises (iii) a kinase inhibitor, preferably selected from the group consisting of an AKT inhibitor, an ATM inhibitor and a DNApK inhibitor.

17. A kit or kit of parts comprising the combination according to any of claims 1 to 16, and, optionally, instructions for use.

18. A combination according to any of claims 1 to 16 for use in a method of treating a SSTR-positive cancer by administering the combination to a cancer patient.

19. The combination for use in a method according to claim 18, wherein the SSTR-positive cancer is an SSTR-2 positive cancer.

20. The combination for use in a method according to claim 18 or 19, wherein the SSTR-positive cancer is a solid cancer.

21. The combination for use in a method according to any of claims 18 to 20, wherein the SSTR-positive cancer is a late stage cancer defined by metastases in the lymph nodes according to a stage GI, GII or GIII cancer.

22. The combination for use in a method according to any of preceding claims 18 to 21, wherein the SSTR-positive cancer is a stage GIV cancer.

23. The combination for use in a method according to any of claims 18 to 22, wherein the SSTR-positive cancer is a neuroendocrine cancer.

24. The combination for use in a method according to any of claims 18 to 23, wherein the SSTR-positive cancer is a neuroendocrine cancer of the gastrointestinal tract, the pancreas or the broncho-pulmonary tract.

25. The combination for use in a method according to any of claims 18 to 24, wherein the neuroendocrine cancer is a pulmonary neuroendocrine cancer.

26. The combination for use in a method according to claim 25, wherein the neuroendocrine pulmonary neuroendocrine cancer is a small-cell lung cancer.

27. The combination for use in a method according to any of claims 18 to 26, wherein the cancer patient is a human at the age of 60 or older.

28. The combination for use in a method according to any of claims 18 to 27, wherein a detection step precedes the treatment step, wherein expression of a Somatostatin receptor on the patient's cancer cells is detected by immune histological staining, Somatostatin receptor scintigraphy, single photon emission computed tomography and/or positron emission tomography.

29. The combination for use in a method according to any of claims 18 to 28, wherein a detection step precedes the treatment step, wherein the detection step includes a step of screening for a gene mutation.

30. The combination for use in a method according to any of claim 29, wherein the gene mutation is selected from the group consisting of a DNA repair gene mutation, more specifically a gene mutation involving homologous recombination repair (HRR), a BRCA1/2, ATM, BARD1, BRIP1, CDK12, CHEK2, FANCL, PALB2, RAD51B, RAD51C, RAD51D, and a RAD54 mutation.

31. The combination for use in a method according to claim 28, wherein the detection step is carried out by single photon emission computed tomography.

32. The combination for use in a method according to claim 28 or 31, wherein the detection step is carried out by the radiopharmaceutical as defined according to any of claims 1 to 16 and wherein the radionuclide of the radiopharmaceutical is suitable for cancer diagnosis.

33. The combination for use in a method according to any of claims 18 to 32, wherein (i) the radiopharmaceutical is administered by a treatment protocol of 1 to 4 administration cycles, preferably 1 to 3 cycles.

34. The combination for use in a method according to any of claims 18 to 33, wherein (ii) the PARP inhibitor is administered by 1 to 4, preferably 2 to 4 administration cycles.

35. The combination for use in a method according to any of claims 18 to 34, wherein the (ii) PARP inhibitor is administered initially and wherein the (i) radiopharmaceutical is administered upon termination of the first PARP inhibitor treatment cycle.

36. The combination for use in a method according to any of claims 18 to 35, wherein administration of the (i) radiopharmaceutical and the (ii) PARP inhibitor is alternating.

37. The combination for use in a method according to any of claims 18 to 36, wherein the (i) radiopharmaceutical is administered intravenously or intratumorally.

38. The combination for use in a method according to any of claims 18 to 37, wherein the (ii) PARP inhibitor is administered orally, intratumorally or intravenously.

39. The combination for use in a method according to any of claims 18 to 38, wherein the (i) radiopharmaceutical is administered intravenously and the (ii) PARP inhibitor is administered orally or intravenously.

40. A radiopharmaceutical as defined by any of claims 1 to 16 for use in a method of treating a patient suffering from an SSTR-positive cancer, whereby the method further comprises the administration of a PARP inhibitor as defined by any of claims 1 to 15 and, optionally, the administration of a kinase inhibitor as defined by claim 16.

41. Radiopharmaceutical as defined by any of claims 1 to 15 for use in a method according to claim 40, wherein the method is as defined by any of claims 18 to 39.

42. A method of treating an SSTR-positive cancer by administering a combination according to any of claims 1 to 16.

43. Method according to claim 42, wherein the method is as defined by any of claims 17 to 38.

44. Method according to claim 42 or 43, wherein the SSTR-positive cancer is a neuroendocrine cancer, preferably a pulmonary neuroendocrine cancer, more preferably small-cell lung cancer.

45. Method according to any of claims 42 to 44, wherein the method comprises a cancer detection step prior to the cancer treatment step, wherein the cancer detection step is preferably carried out in vivo by single photon emission computed tomography or positron emission tomograph and/or preferably carried out by a step of screening for a gene mutation.

46. Method according to claim 45, wherein the gene mutation is selected from the group consisting of a DNA repair gene mutation, more specifically a gene mutation involving homologous recombination repair (HRR), a BRCA1/2, ATM, BARD1, BRIP1, CDK12, CHEK2, FANCL, PALB2, RAD51B, RAD51C, RAD51D, and a RAD54 gene mutation.

47. Method according to any of claims 42 to 46, wherein the SSTR-positive cancer is as defined by any of claims 19 to 26.
